SEQUENCE LISTING (16 sequences)

SEQ ID NO: 1 - RNA, 21 nt, Artificial Sequence
  Description of Artificial Sequencechemically synthesized siRNA
aagcucuggaacuaccaacga

SEQ ID NO: 2 - RNA, 21 nt, Artificial Sequence
  Description of Artificial Sequencechemically synthesized siRNA
ucguugguaguuccagagcuu

SEQ ID NO: 3 - DNA, 3392 bp, Homo sapiens
actccctcactggctgccattgaaagagtccacttctcagtgactcctagctgggcactggatgcagttgaggattgctggtcaatatgattcttcttgctgtgctttttctctgcttcatttcctcatattcagcttctgttaaaggtcacacaactggtctctcattaaataatgaccggctgtacaagctcacgtactccactgaagttcttcttgatcggggcaaaggaaaactgcaagacagcgtgggctaccgcatttcctccaacgtggatgtggccttactatggaggaatcctgatggtgatgatgaccagttgatccaaataacgatgaaggatgtaaatgttgaaaatgtgaatcagcagagaggagagaagagcatcttcaaaggaaaaagcccatctaaaataatgggaaaggaaaacttggaagctctgcaaagacctacgctccttcatctaatccatggaaaggtcaaagagttctactcatatcaaaatgaggcagtggccatagaaaatatcaagagaggtctggctagcctatttcagacacagttaagctctggaaccaccaatgaggtagatatctctggaaattgtaaagtgacctaccaggctcatcaagacaaagtgatcaaaattaaggccttggattcatgcaaaatagcgaggtctggatttacgaccccaaatcaggtcttgggtgtcagttcaaaagctacatctgtcaccacctataagatagaagacagctttgttatagctgtgcttgctgaagaaacacacaattttggactgaatttcctacaaaccattaaggggaaaatagtatcgaagcagaaattagagctgaagacaaccgaagcaggcccaagattgatgtctggaaagcaggctgcagccataatcaaagcagttgattcaaagtacacggccattcccattgtggggcaggtcttccagagccactgtaaaggatgtccttctctctcggagctctggcggtccaccaggaaatacctgcagcctgacaacctttccaaggctgaggctgtcagaaacttcctggccttcattcagcacctcaggactgcgaagaaagaagagatccttcaaatactaaagatggaaaataaggaagtattacctcagctggtggatgctgtcacctctgctcagacctcagactcattagaagccattttggactttttggatttcaaaagtgacagcagcattatcctccaggagaggtttctctatgcctgtggatttgcttctcatcccaatgaagaactcctgagagccctcattagtaagttcaaaggttctattggtagcagtgacatcagagaaactgttatgatcatcactgggacacttgtcagaaagttgtgtcagaatgaaggctgcaaactcaaagcagtagtggaagctaagaagttaatcctgggaggacttgaaaaagcagagaaaaaagaggacaccaggatgtatctgctggctttgaagaatgccctgcttccagaaggcatcccaagtcttctgaagtatgcagaagcaggagaagggcccatcagccacctggctaccactgctctccagagatatgatctccctttcataactgatgaggtgaagaagaccttaaacagaatataccaccaaaaccgtaaagttcatgaaaagactgtgcgcactgctgcagctgctatcattttaaataacaatccatcctacatggacgtcaagaacatcctgctgtctattggggagcttccccaagaaatgaataaatacatgctcgccattgttcaagacatcctacgtttggaaatgcctgcaagcaaaattgtccgtcgagttctgaaggaaatggtcgctcacaattatgaccgtttctccaggagtggatcttcttctgcctacactggctacatagaacgtagtccccgttcggcatctacttacagcctagacattctctactcgggttctggcattctaaggagaagtaacctgaacatctttcagtacattgggaaggctggtcttcacggtagccaggtggttattgaagcccaaggactggaagccttaatcgcagccacccctgacgagggggaggagaaccttgactcctatgctggtatgtcagccatcctctttgatgttcagctcagacctgtcacctttttcaacggatacagtgatttgatgtccaaaatgctgtcagcatctggcgaccctatcagtgtggtgaaaggacttattctgctaatagatcattctcaggaacttcagttacaatctggactaaaagccaatatagaggtccagggtggtctagctattgatatttcaggtgcaatggagtttagcttgtggtatcgtgagtctaaaacccgagtgaaaaatagggtgactgtggtaataaccactgacatcacagtggactcctcttttgtgaaagctggcctggaaaccagtacagaaacagaagcaggcttggagtttatctccacagtgcagttttctcagtacccattcttagtttgcatgcagatggacaaggatgaagctccattcaggcaatttgagaaaaagtacgaaaggctgtccacaggcagaggttatgtctctcagaaaagaaaagaaagcgtattagcaggatgtgaattcccgctccatcaagagaactcagagatgtgcaaagtggtgtttgcccctcagccggatagtacttccagcggatggttttgaaactgacctgtgatattttacttgaatttgtctccccgaaagggacacaatgtggcatgactaagtacttgctctctgagagcacagcgtttacatatttacctgtatttaagatttttgtaaaaagctacaaaaaactgcagtttgatcaaatttgggtatatgcagtatgctacccacagcgtcattttgaatcatcatgtgacgctttcaacaacgttcttagtttacttatacctctctcaaatctcatttggtacagtcagaatagttattctctaagaggaaactagtgtttgttaaaaacaaaaataaaaacaaaaccacacaaggagaacccaattttgtttcaacaatttttgatcaatgtatatgaagctcttgataggacttccttaagcatgacgggaaaaccaaacacgttccctaatcaggaaaaaaaaaaaaaaaaaaaagtaagacacaaacaaaccatttttttctctttttttggagttgggggcccagggagaagggacaaggcttttaaaagacttgttagccaacttcaagaattaatatttatgtctctgttattgttagttttaagccttaaggtagaaggcacatagaaataacatc

SEQ ID NO: 4 - protein, 894 aa, Homo sapiens
MILLAVLFLCFISSYSASVKGHTTGLSLNNDRLYKLTYSTEVLLDRGKGKLQDSVGYRISSNVDVALLWRNPDGDDDQLIQITMKDVNVENVNQQRGEKSIFKGKSPSKIMGKENLEALQRPTLLHLIHGKVKEFYSYQNEAVAIENIKRGLASLFQTQLSSGTTNEVDISGNCKVTYQAHQDKVIKIKALDSCKIARSGFTTPNQVLGVSSKATSVTTYKIEDSFVIAVLAEETHNFGLNFLQTIKGKIVSKQKLELKTTEAGPRLMSGKQAAAIIKAVDSKYTAIPIVGQVFQSHCKGCPSLSELWRSTRKYLQPDNLSKAEAVRNFLAFIQHLRTAKKEEILQILKMENKEVLPQLVDAVTSAQTSDSLEAILDFLDFKSDSSIILQERFLYACGFASHPNEELLRALISKFKGSIGSSDIRETVMIITGTLVRKLCQNEGCKLKAVVEAKKLILGGLEKAEKKEDTRMYLLALKNALLPEGIPSLLKYAEAGEGPISHLATTALQRYDLPFITDEVKKTLNRIYHQNRKVHEKTVRTAAAAIILNNNPSYMDVKNILLSIGELPQEMNKYMLAIVQDILRLEMPASKIVRRVLKEMVAHNYDRFSRSGSSSAYTGYIERSPRSASTYSLDILYSGSGILRRSNLNIFQYIGKAGLHGSQVVIEAQGLEALIAATPDEGEENLDSYAGMSAILFDVQLRPVTFFNGYSDLMSKMLSASGDPISVVKGLILLIDHSQELQLQSGLKANIEVQGGLAIDISGAMEFSLWYRESKTRVKNRVTVVITTDITVDSSFVKAGLETSTETEAGLEFISTVQFSQYPFLVCMQMDKDEAPFRQFEKKYERLSTGRGYVSQKRKESVLAGCEFPLHQENSEMCKVVFAPQPDSTSSGWF

SEQ ID NO: 5 - DNA, 2878 bp, Mus musculus
ctggatgtggcagagggagccagcatgatcctcttggcagtgctttttctctgcttcttctcctcctactctgcttccgttaaaggtcacacaactggcctctcattaaataatgagcggctatacaagctcacgtactccactgaagtgtttcttgatgggggcaaaggaaaaccgcaagacagcgtgggctacaaaatctcatctgatgtggacgttgtgttactgtggaggaatcctgatggtgatgatgatcaagtgatccaagtcacgataacagctgttaacgttgaaaatgcgggtcaacagagaggcgagaagagcatcttccagggcaaaagtacacctaagatcatagggaaggacaacctggaggctctgcagagacccatgcttcttcatctggtccgggggaaggtcaaggagttctactcctatgaaaacgagccagtgggcatagaaaatctcaagagaggcttggctagcttattccagatgcagctaagctctggaactaccaacgaggtagatatctctggggattgtaaagtgacctaccaggcccaacaagacaaagtggtcaaaattaaggctctggatacatgcaaaattgagcggtctggatttacaacggcaaaccaggtgctgggcgtcagttcaaaagccacatctgtcactacctacaagatagaggacagctttgtcaccgctgtgcttgcagaagagaccagggcttttgccttgaacttccaacaaaccatagcaggaaaaatagtgtcaaagcagaaattggagctgaagacaactgaagccggcccaaggatgatccccgggaagcaagtggcaggtgtaattaaagcagttgattccaaatacaaagccattcccattgtgggacaggtcctcgagcgtgtctgcaaaggatgcccttctctggcggagcactggaagtccatcagaaagaacctggagcctgaaaacctgtccaaggccgaggctgtccagagcttcctggccttcatccagcacctccggacttcgaggagagaagagatcctccagattctgaaggcagagaagaaagaagtgctccctcagctggtggatgccgtcacctctgctcagactccagactcgctagaagccatcctggactttttggatttcaaaagtgacagcagtatcatactccaggaaaggttcctctatgcctgtggctttgccacccaccctgatgaagaactcctacgagccctccttagtaagttcaaaggttcctttgcaagcaacgacatcagagagtcggttatgatcatcattggagccctagtcaggaagctgtgtcagaatgaaggctgcaagctcaaggcagtggtggaagctaagaagctgatcctgggaggacttgaaaaaccagagaagaaagaagacaccacaatgtacctgctggccctgaagaatgccttgcttcccgaaggcatcccgctccttctgaagtatgctgaggctggagaagggcccgtcagccacctggccaccactgttctccagagatacgatgtctccttcatcacagatgaggtgaagaagaccttgaacaggatataccaccagaatcgtaaggttcatgagaagacggtgcgcacaactgccgctgctgtcatcttaaagaacccatcctacatggatgtgaagaacatcctgctgtccattggggaactcccgaaagagatgaacaaatacatgctcaccgttgtgcaagacatcctgcattttgaaatgcctgcaagcaaaatgatccgtcgagttctcaaggagatggctgttcacaattatgaccgtttctccaagagtggatcctcttctgcctatactggctacgtagaacgtagcccccgtgcagcgtccacatacagccttgacatcctttactctggctctggcattctgaggagaagtaacctgaacatcttccagtacatcaaaggaacagagcttcatggtagtcaggtggtgattgaagcccaagggctggaaggcttaattgcagccactcctgatgaaggagaggagaaccttgactcttatgctggcatgtcagccatcctgtttgatgttcagcttaggcctgtcacattttttaatggatacagtgatttgatgtccaaaatgctgtcggcatccggcgaccctgtcagcgtggtgaaagggcttattctgttaatagaccattctcaggatattcagctgcaatctggactaaaggccaatatggagatccagggtggtctagctattgatatttctggttcaatggaattcagtctgtggtatcgcgagtctaaaacccgagtgaaaaatcgggtggctgtggtgataaccagcgacgtcacagtggatgcctcttttgtgaaagctggtctggaaagcagagcggagacagaggctgggctggagttcatctccacagtgcagttctcacagtacccgttcttggtctgcatgcagatggacaaggctgaagccccactcaggcaattcgagacaaagtatgaaaggctatctacaggcaggggatatgtctctcggagaagaaaagagagcctagtggccggatgtgaactccccctccatcaacagaactctgagatgtgcaacgtggtattcccacctcagccagaaagcgataactccggtggatggttttgattcccgtgggttcccttccaccagaacgatatgctatgacgtgcctgacccttgctctctgagagcacagtgtttacatatttacctgtatttaagatgtttgtaaagagcagtggagaacttcagttgattaaagttgaacctattcaggagaagacccacagtgtcc

SEQ ID NO: 6 - protein, 894 aa, Mus musculus
MILLAVLFLCFFSSYSASVKGHTTGLSLNNERLYKLTYSTEVFLDGGKGKPQDSVGYKISSDVDVVLLWRNPDGDDDQVIQVTITAVNVENAGQQRGEKSIFQGKSTPKIIGKDNLEALQRPMLLHLVRGKVKEFYSYENEPVGIENLKRGLASLFQMQLSSGTTNEVDISGDCKVTYQAQQDKVVKIKALDTCKIERSGFTTANQVLGVSSKATSVTTYKIEDSFVTAVLAEETRAFALNFQQTIAGKIVSKQKLELKTTEAGPRMIPGKQVAGVIKAVDSKYKAIPIVGQVLERVCKGCPSLAEHWKSIRKNLEPENLSKAEAVQSFLAFIQHLRTSRREEILQILKAEKKEVLPQLVDAVTSAQTPDSLEAILDFLDFKSDSSIILQERFLYACGFATHPDEELLRALLSKFKGSFASNDIRESVMIIIGALVRKLCQNEGCKLKAVVEAKKLILGGLEKPEKKEDTTMYLLALKNALLPEGIPLLLKYAEAGEGPVSHLATTVLQRYDVSFITDEVKKTLNRIYHQNRKVHEKTVRTTAAAVILKNPSYMDVKNILLSIGELPKEMNKYMLTVVQDILHFEMPASKMIRRVLKEMAVHNYDRFSKSGSSSAYTGYVERSPRAASTYSLDILYSGSGILRRSNLNIFQYIKGTELHGSQVVIEAQGLEGLIAATPDEGEENLDSYAGMSAILFDVQLRPVTFFNGYSDLMSKMLSASGDPVSVVKGLILLIDHSQDIQLQSGLKANMEIQGGLAIDISGSMEFSLWYRESKTRVKNRVAVVITSDVTVDASFVKAGLESRAETEAGLEFISTVQFSQYPFLVCMQMDKAEAPLRQFETKYERLSTGRGYVSRRRKESLVAGCELPLHQQNSEMCNVVFPPQPESDNSGGWF

SEQ ID NO: 7 - DNA, 21 bp, Artificial Sequence
  Description of Artificial Sequence oligonucleotide primer
ggagaaacggtcataattgtg

SEQ ID NO: 8 - DNA, 21 bp, Artificial Sequence
  Description of Artificial Sequence oligonucleotide primer
gtgggccgctctaggcaccaa

SEQ ID NO: 9 - DNA, 24 bp, Artificial Sequence
  Description of Artificial Sequence oligonucleotide primer
ctctttgatgtcacgcacgatttc

SEQ ID NO: 10 - DNA, 25 bp, Artificial Sequence
  Description of Artificial Sequence oligonucleotide primer
ggactttttggatttcaaaagtgac

SEQ ID NO: 11 - protein, 265 aa, Homo sapiens
  VARIANT (1)..(261): Wherein Xaa is any amino acid.
MDPPRPALLALLAXPXLLLLLLAGARXEEEXLENVXLVCPKDXTRFXHLXKXXTYNYEAESSSGVPGTAXSRSATRXNCKXELEVPQLCSFILKXSQCTLKEVYGFNPEGKALLKKTKNSXEXAAAMSRXELKLAIPEGKQVFLYPEKDEPTYILNIKRGIISALLVPPEXEEAKQXLFXDTVYGNCSTHFTVKTRXGNXATXXSTERDLGQCDRFKPIRTGISPXALIKGMXRPLSTLIXSXQSCQXTLDAKRKHVAEAXCKEQ

SEQ ID NO: 12 - protein, 335 aa, Homo sapiens
  VARIANT (1)..(335): Wherein Xaa is any amino acid.
MGCLLFLLLWALLQAWGSAEVPQRLFPLRCLQISSFANSSWTRTDGLAWLGELQTHXWSNDSDTVRXXKPWSQGTFSDQQWETLQHIFRVYRSSFTXDXKEXAKXXRLSYPLELQXSAGCEXHPGNASNNFFHVAFQGKDILSFQGTSXEPXQEAPXWVNLAXQXLNQDKWTXETXQWLLNGTCPQFVSGLLESGKSELKKQVKPKXWLSRGPXPXPGRLLLXCHVSGXYPKPVWVKWXXGEQEQQGTQPXDXXPNXDETWYLRATLXVXAGEAXGLSCRVKHSSLXGQDIVLYWGGSYTSMGLIALAVLACLLFLLIVGFTSRFKRQTSYQGVL

SEQ ID NO: 13 - protein, 210 aa, Homo sapiens
  VARIANT (1)..(210): Wherein Xaa is any amino acid.
KCVQSXKPSLMIQKAXXQALRKMEPKDKDQEVLLQTFLDDASPGDXRXAAXLMXXRSPSQADXNKIVQXLPWEQNEQVKNXVAXHIANXLNSEEXDXQDLKKLVXEAXKESQLPTVMDFRKFSRNYQLYKSVXLPSLDPXSXKIEGNLXFDPNNXLPKESMXXTTLTAFGFASXDXXEIXLEGKGFEPTLXAXFGKQXFFPXSVNKALYW

SEQ ID NO: 14 - protein, 301 aa, Homo sapiens
  VARIANT (1)..(301): Wherein Xaa is any amino acid.
FSYNNKYGMVAQVTQTLKLEDTPKINSRFFGEGTXKMGLAXESTKSTSPPKXAEAVXXXLQELKKLTISXQXIQRAXLFNXXVTELRGLSDEAVTSXLPQLIEXSSPXXLQALVQCGXPQCSTHIXQXLKXVHANPLLIDVVTYLVALXPEPSAQQXREIFNMARXQRSRATLYALSHAVNNYHKXNPXGTQELXDIANXLMEQIQDDCXGDEDYTYLXLRXIGNMGQTMEQLTPELKSXILKCVQSTKPSXXIQKAAIQXLRKMEPKDKDQXXLLQTFLDDASPGDKRLAAYLMLXRSPS

SEQ ID NO: 15 - protein, 335 aa, Homo sapiens; identical to SEQ ID NO: 12
  VARIANT (1)..(335): Wherein Xaa is any amino acid.

SEQ ID NO: 16 - protein, 335 aa, Homo sapiens
  VARIANT (1)..(335): Wherein Xaa is any amino acid.
MGCLLFLLLWALLQAWGSAEVPQRLFPLRCLQISSFANSSWTXTDGLAXLGELQTHSWSXDSDTXXXLKPWSQGTFSXQXWETLXHIFXXYRSSFTRDVKEFAKXLRLSYPXELQXXAGCEVHPGXASNNFFHXAXQGXDILSFQGTSWEPTQEAPXWVNLAIQXLNQDKWTRXTVQWLLNGTCPQFVSGLLEXGKXELKKQXKPKAXLSRGPSPGPGRLLLVCHVXGFYPKPVWXKWXRGEQEQQGTQPGDILPNXDETWYLRATLDXXAGEAAGLXCRVKHSSLEGQXXXLYWGGSYTSMGLIALAVLACLXFLLIVGFTSRFKRQTSYQGVL

(19) United States
(12) Patent Application Publication
(10) Pub. No.: US 2005/0090426 A1
(43) Pub. Date: Apr. 28, 2005

(54) Methods of inhibiting inflammation

(76) Inventor: Richard S. Blumberg, Chestnut Hill, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/808,052

(22) Filed: Mar. 24, 2004

Related U.S. Application Data
(60) Provisional application No. 60/457,048, filed on Mar. 24, 2003.

(51) Int. Cl. A61K 38/17

(52) U.S. Cl. 514/2

(57) ABSTRACT

The invention provides a method of inhibiting inflammation in a mammal, by administering to the mammal composition containing a compound which inhibits the expression or activity of a microsomal triglyceride transfer protein.

### RELATED APPLICATIONS

This application claims priority to U.S. Ser. No. 60/457,048 filed Mar. 24, 2003 which is incorporated herein by reference in its entirety.

### STATEMENT AS TO FEDERALLY SPONSORED RESEARCH

This invention was made with U.S. government support under NIH grants DK44319, DK53056 and DK51362. The government has certain rights in the invention.

### FIELD OF THE INVENTION

The invention relates to the control of inflammation.

### BACKGROUND OF THE INVENTION

The inflammatory response plays an important role in limiting and controlling pathogenic infections. Elevated levels of proinflammatory cytokines are also associated with a number of diseases of autoimmunity such as toxic shock syndrome, rheumatoid arthritis, osteoarthritis, diabetes and inflammatory bowel disease. In these diseases, chronic elevation of inflammation exacerbates or causes much of the pathophysiology observed.

### SUMMARY OF THE INVENTION

The invention is based on the discovery that a decrease in microsomal triglyceride transfer protein (MTP), leads to an inhibition of inflammation in a mouse model for clinical inflammation. Accordingly, the invention features methods of preventing or inhibiting inflammation in a bodily tissue. The inflammation is CD1-mediated inflammation. Inflammation is inhibited by administering to an inflamed tissue (or a tissue that is at risk of becoming inflamed) a MTP inhibitor. An inflamed tissue is characterized by redness, pain and swelling of the tissue. The tissue includes epithelial tissue or liver tissue. For example, the epithelial tissue is intestinal tissue or skin. The invention also features methods of preventing or alleviating a symptom of an inflammatory disorder or a CD1-mediated immunopathology in a subject by identifying a subject suffering from or at risk of developing an inflammatory disorder or a CD1-mediated immunopathology and administering to the subject a MTP inhibitor. A CD1-mediated immunopathology includes for example an autoimmune disorder such as diabetes, colitis or hepatitis.

Inflammation is inhibited by contacting a cell with a MTP inhibitor in an amount that leads to a reduction in the production of a proinflammatory or inflammatory cytokine or in an amount that leads to inhibition of T-cell activation, e.g. activation of a CD1d restricted T cell. The cell is any cell that is capable of expressing MTP, e.g., an antigen presenting cell such as a B-cell, monocyte, macrophage, dendritic cell; a hepatocyte; or an epithelial cell such as an intestinal epithelial cell. Optimally, the cell expresses CD1 (e.g., CD1a, CD1b, CD1c, CD1d or CD1e), a natural killer receptor, or an invariant T-cell receptor (e.g., Vα24Jα15). For example, the cell overexpresses MTP compared to a level of expression associated the normal non-inflamed tissue or cells. The cell is contacted in vivo, in vitro, or ex vivo. Inflammatory cytokines include for example, interferon, interleukin, or tumor necrosis factor alpha.

The invention methods of inhibiting antigen presentation or cell surface expression of CD1 (e.g., CD1a, CD1b, CD1c, CD1d or CD1e) by contacting a cell with a MTP inhibitor. CD 1 mediated antigen presentation is inhibited such that the amount of lipid associated with CD1 or the amount of binding to CD1 us reduced in the presence of the inhibitor compared to the absence of the inhibitor. The cell is an immune cell such as an antigen presenting cell. The cell expresses CD1. For example, the cell express CD1d. The cell is a B-cell, a macrophage, a dendritic cell a hepatocyte an epithelial cell or any cell expressing CD1.

Also included in the invention is a method of inhibiting an association of MTP and CD1 by contacting a cell with an MTP-binding compound or a CD1 binding compound such that the association is reduced in the presence of the compound compared to the absence of the compound. The association is non-covalent such as a van der Walls interaction, hydrogen bonding, electrostatic interaction, or hydrophobic interaction. A MTP binding compound is any compound that interacts (covalently or non-covalently) with an MTP protein. The MTP binding compound decreases the lipid transfer activity of the MTP protein. The compound interacts with the M subunit of an MTP protein. Alternatively, the compound interacts with the P subunit of an MTP protein. For example, the compound interacts with the lipid transfer domain, the membrane associating domain or apoB binding domain of the M subunit. Optimally, the compound interacts with the A or C β-sheets or both of the M subunit. The compound interacts with the amino acid residue at position 780 of the M subunit of a MTP protein. A CD1 binding compound is any compound that interacts with a CD1 polypeptide. The CD1 binding compound prevents lipidation of the CD1 polypeptide. The compound interacts with the α1, α2, α3 or the β2 m domain of a CD1 polypeptide. For example, the compound interacts with residues or regions of the CD1 molecule that share homology with the MTP binding domains on apoB as shown in FIGS. 12-14, e.g. α1 and α2 domains that contain lipid contact sites.

A MTP inhibitor is a compound which decreases the expression or activity of MTP. MTP activities include binding to CD1 and transferring lipid. Activity of MTP is measured by determining the transfer of lipids from HDL to LDL. For example, a decrease in the transfer of lipid from HDL to LDL in the presence of the compound (compared to the amount detected in the absence of the compound) indicates a reduction of MTP activity. Methods of measuring transfer of lipid are well known in the art.

The subject is a mammal such as human, a primate, mouse, rat, dog, cat, cow, horse, pig. The subject is suffering from or at risk of developing an inflammatory disorder. Inflammatory disorders include, cardiovascular inflammation, gastrointestinal inflammation, hepatic inflammation, pulmonary inflammation, autoimmune disorders or skeletal inflammation. A subject suffering from or at risk of developing inflammatory is identified by methods known in the art, e.g., gross examination of tissue or detection of inflammation associated in tissue or blood. Symptoms of inflammation include pain, redness and swelling of the affected tissue. A subject suffering from gastrointestinal inflammation, such as colitis, is identified histologically by the presence of mucosal necrosis or hemorrhagic lesions in the colon, frequent diarrhea or blood and pus in the stool.

MTP inhibitors inhibitors include compounds that reduces expression of a Mttp gene product. For example, the compound is an antisense MTP nucleic acid, an MTP-specific short-interfering RNA, or a MTP specific ribozyme. Alternatively, the MTP inhibitor is a protease inhibitor, a carboline compound or a benzimidazole-based analogue. MTP inhibitors are administered alone or in combination with another anti-inflammatory agent or therapeutic drugs used to treat an inflammatory disorder. For example, the MTP inhibitor is administered in combination with corticosteroids, cyclosporine, nicotine, or heparin.

Other features and advantages of the invention will be apparent from the following detailed description and from the claims.

### BRIEF DESCRIPTION OF THE DRAWINGS

FIG. 1A is a photograph of a Western blot. Lysates of hepatocytes from wild-type C57BL/6J (B6) mice were immunoprecipiated with control immunoglobulins (rat IgG2b, mouse IgG1), an anti-H-2D antibody (8F12, mouse IgG1), or an anti-CD1d antibody (1B1, rat IgG2b) and the immunoprecipitates Western blotted an anti-MTP antibody (left panel) or a rabbit anti-MHC class I serum (right panel)

FIG. 1B is a photograph of a Western blot. Lysates were prepared from B6 or MTP^{flox/flox}Mx1Cre mice before or after pIpC treatment and subjected to the same analysis as described in panel A.

FIG. 1C is a photograph of hepatocytes isolated from MTP^{flox/flox}Mx1Cre mice prior to treatment with pIpC or MTP^{flox/flox}Mx1Cre mice at one day after completion of pIpC treatment (bottom panels) were stained with the anti-CD 1d mAb, 1B1, an anti-ER antibody and a marker for the nuclei. MTP^{flox/flox}Mx1Cre mice that were treated with pIpC were also stained for expression of MHC class I using an H-2K^{b} antibody.

FIG. 1D is an illustration depicting the results of fluorescent activated cell sorting showing a decrease in CD1d surface expression on hepatocytes following deletion of the MTP gene.

FIG. 2A is a bar chart showing induction of mIL-2 secretion by the mouse iNKT cell hybridoma DN32.D3 cocultivated with hepatocytes, obtained from MTP^{flox/flox}Mx1Cre mice treated (+) or not treated (−) with pIpC, in the presence of either αGalCer (+, 100 ng/ml) or vehicle control (−). (n=6 mice per group). ND, not detectable. *, P<0.001.

FIG. 2B is a bar chart showing induction of mIL-2 production by CD11c⁺ liver cells from MTP^{flox/flox}Mx1Cre mice treated with or without pIpC that were cultured for 24 h with CD4⁺ splenocytes from OT-II mice and 100 μg/ml OVA or PBS. In the presence of OVA, the concentration of IL-2 in culture supernatants from CD11c⁺cells isolated from pIpC-treated mice was significantly increased consistent with an upregulation of MHC class II by interferon-α. **, P<0.005.

FIG. 2C is a bar chart showing induction of mIL-2 production by hepatocytes from MTP^{flox/flox}Mx1Cre mice treated (+) or not treated (−) with pIpC that were cocultivated with the autoreactive mouse CD1d-restricted T cell hybridomas 14S.6 (non-invariant TCR-α chain) or 24.8 (invariant TCR-α chain) (n=2 mice per group, duplicate cultures). CD1d-restriction was confirmed with an anti-CD1d blocking antibody (3C11). Note the reduction in the degree of autoreactivity of the hybridomas towards hepatocytes derived from pIpC treated vs. non-pIpC treated MTP^{flox/flox}Mx1Cre mice and the blockade of autoreactivity with the 3C11 mAb. *, P<0.001.

FIG. 2D is a series of bar charts showing serum ALT levels (U/L) (n=3 mice per group) from C57BL/6J mice (panels a and b) or MTP^{flox/flox}Mx1Cre mice (panels c and d) Mice were treated with either vehicle alone (open bars), pIpC alone (closed bars), αGalCer alone (hatched bars) or pIpC followed by αGalCer (shaded bars). Panels a and c show the results when αGalCer was administered at day 1 after completion of the pIpC treatment regimen and panels b and d when αGalCer was administered at day 10 after similar treatment. Similar results were obtained when serum AST levels were assessed (*; P<0.05; **,p<0.01.

FIG. 2E are photographs of gross examination of the livers of C57BL/6J mice (panels a and b) and MTP^{flox/flox}Mx1Cre mice (panels c and d) that received αGalCer with (panels b and d) or without (panels a and c) prior pIpC treatment. Note the multiple foci of liver necrosis in all groups (white arrows) except for the MTP^{flox/flox}Mx1Cre mice that received pIpC treatment at 10 days prior to αGalCer administration (panel d). The latter group of mice only exhibited a pale liver consistent with the lipid accumulation.

FIG. 2F are photographs of histopathologic analysis livers of αGalCer induced hepatitis in C57BL/6J and MTP^{flox/flox}Mx1Cre mice treated as described in FIG. 2C.

FIG. 3A depicts the results of silencing the MTPp gene in intestinal epithelial cells. Panel A are photographs of blots showing MTP transcripts in MODE-K cells relative to hepatocytes. Panel B are photographs of blots showing MTP transcripts in MODE-K cells that were either not treated (−) or treated with mock siRNA oligomers specific for an irrelevant gene target (Src homology domain 2 phosphatase 1, SHP-1; mock) or siRNA oligomers specific for MTP (silenced). β-actin uses as a control. Panel C is a bar chart showing quantitation of the ratios between MTPp and β-actin levels for each of the experimental conditions shown in Panel B. *, P<0.001.

FIG. 3B is a bar chart showing MODE-K cell (treated in triplicate as in FIG. 2A with siRNA oligomers specific for SHP-1 (mock) or MTP) induction of mIL-2 production upon by cocultivation with the DN32.D3 cell line in the presence of αGalCer (+, 100 ng/ml) and mIL-2 production (pg/ml) assessed. **, P<0.01. ND, non-detectable.

FIG. 4A are line graphs showing wasting as defined by % of body weight from baseline of B6 (panel A) or MTP^{flox/flox}Mx1Cre (panel B) of mice subjected to oxazolone colitis treated either with vehicle (squares) or with pIpC (circles) prior to skin painting with oxazolone followed by rectal challenge with either 50% ethanol alone as a control (open squares and circles) or 50% ethanol containing oxazolone (closed circles and squares). Data are shown as mean values±SEM and represent 12 mice per group. *, P<0.005; **, P<0.001 for MTP^{flox/flox}Mx1Cre mice with or without pIpC treatment.

FIG. 4B are photographs of H & E stained microscopic images from oxazolone colitis or ethanol control group in WT B6 or MTP^{flox/flox}Mx1Cre mice treated with vehicle or pIpC are shown (magnification; ×100). One representative picture from each group is shown. (Panel A, B6 mice with oxazolone+vehicle; Panel B, B6 mice with oxazolone+pIpC; Panel C, MTP^{flox/flox}Mx1Cre mice with oxazolone+vehicle; and Panel D, MTP^{flox/flox}Mx1Cre mice with oxazolone+pIpC). Black arrow in Panel D indicates mild inflammation with minimal epithelial cell proliferation in MTP^{flox/flox}Mx1Cre mice treated with pIpC in comparison to the untreated control group (Panel C).

FIG. 4C are bar charts showing the quantitative histopathological assessment of oxazolone colitis activity in B6 (Panel A) or MTP^{flox/flox}Mx1Cre (Panel B) mice from the following groups are shown: ethanol control, shaded bars; oxazolone, open bars; ethanol with pIpC, hatched bars; oxazolone with pIpC, filled bars. Note the significant suppression of oxazolone colitis in MTP^{flox/flox}Mx1Cre but not B6 mice pretreated with pIpC (filled bars) in comparison to vehicle treated animals (open bars) *; P<0.001. Data are shown as mean values±SEM and represent ten mice per group.

FIG. 5 is a diagram illustrating an experimental model for reduction of oxazolone-induced colitis in MTP deficient mice.

FIG. 6 is a photograph of a Western Blot showing MTP expression in antigen presenting cell.

FIG. 7 is a bar chart showing antigen presentation of siRNA silenced and wild type U397 cells at an effecter target ration of 1:1.

FIG. 8 is a bar chart showing antigen presentation of siRNA silenced and wild type U397 cells at an effecter target ration of 10:1.

FIG. 9 is a bar chart showing antigen presentation of siRNA silenced and wild type U397 cells at an effecter target ration of 100:1.

FIG. 10 is an illustration depicting types of natural killer T-cells.

FIG. 11 is an illustration depicting the regulatory functions of iNKT cells.

FIG. 12 is an illustration depicting a sequence alignment of the low affinity MTP binding region (amino acids 1-269) of apoB.

FIG. 13 is an illustration depicting a sequence alignment of the high affinity MTP binding region (amino acids 512-721) of apoB.

FIG. 14 is an illustration depicting a sequence alignment of the high affinity MTP binding region (amino acids 270-570) of apoB.

### DETAILED DESCRIPTION

The invention is based in part on the discovery that a decrease in microsomal triglyceride transfer protein (MTP) expression results in an anti-inflammatory effect.

Activation of CD1d-restricted T cells is involved in the regulation of immune-mediated inflammatory disorders, anti-tumor immunity, and anti-microbial immunity, among other conditions, through the ability of CD1d to present endogenous and/or foreign lipids to iNKT cells.

MTP, an endoplasmic reticulum (ER) resident protein in hepatocytes and intestinal epithelial cells (IEC) is essential for lipidation of apolipoprotein-B (apo-B). Specifically, MTP catalyzes the transport of triglyceride (TG), cholesteryl ester (CE), and phosphatidylcholine (PC) between small unilamellar vesicles (SUV). MTP is a complex of two subunits of molecular weights 58,000 (the “P⇄ subunit) and 88,000 (the “M” subunit. The P subunit is inactive with respect to its isomerase activity in the MTP complex. The P subunit is not essential for its lipid transfer activity, whereas the M subunit is essential for the transfer of lipid. MTP contains three structural motifs (i.e., N-terminal β-barrel, central α-helix, and C-terminal lipid cavity) and three functional domains (i.e., lipid transfer, membrane associating and apoB binding). The lipid transfer domain is involved in loading and unloading of lipid molecules necessary for lipid transfer. The lipid binding domain are formed by the A and C β-sheets of the M subunit. A non-sense mutation in the A sheet at amino acid residue 780 (Asn-Tyr) abolishes MTP's lipid binding activity. In addition, to the lipid transfer activity function, MTP has been shown to physically interact with apoB and this association is important in the regulation of lipoprotein production. However, the lipid transfer domain is different from the apo-B binding domain as lipid transfer inhibitors do not inhibit apo-B-MTP binding.

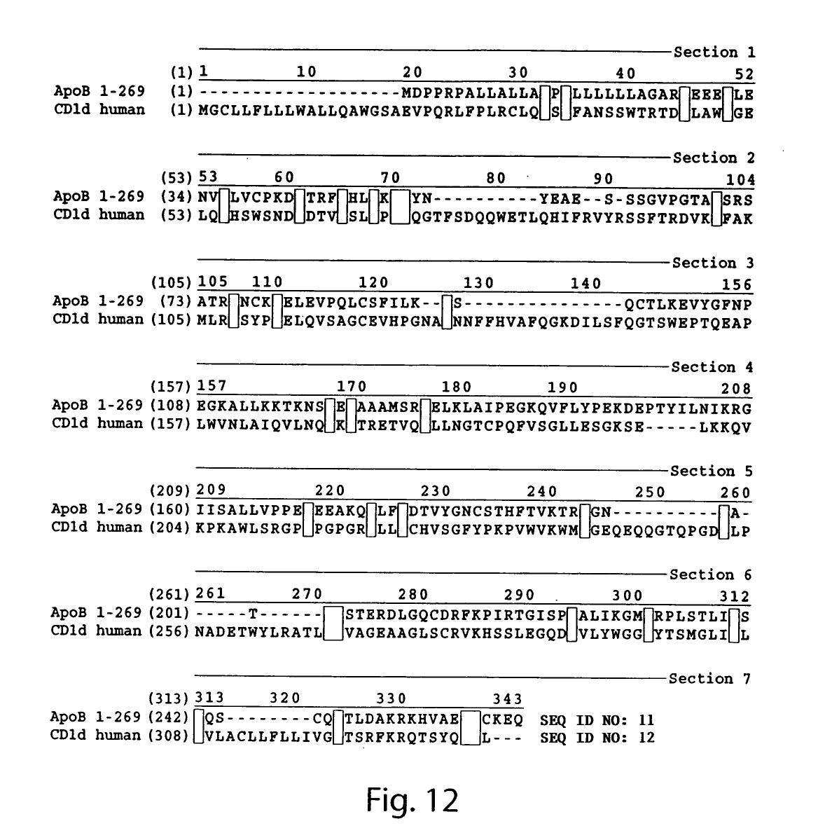

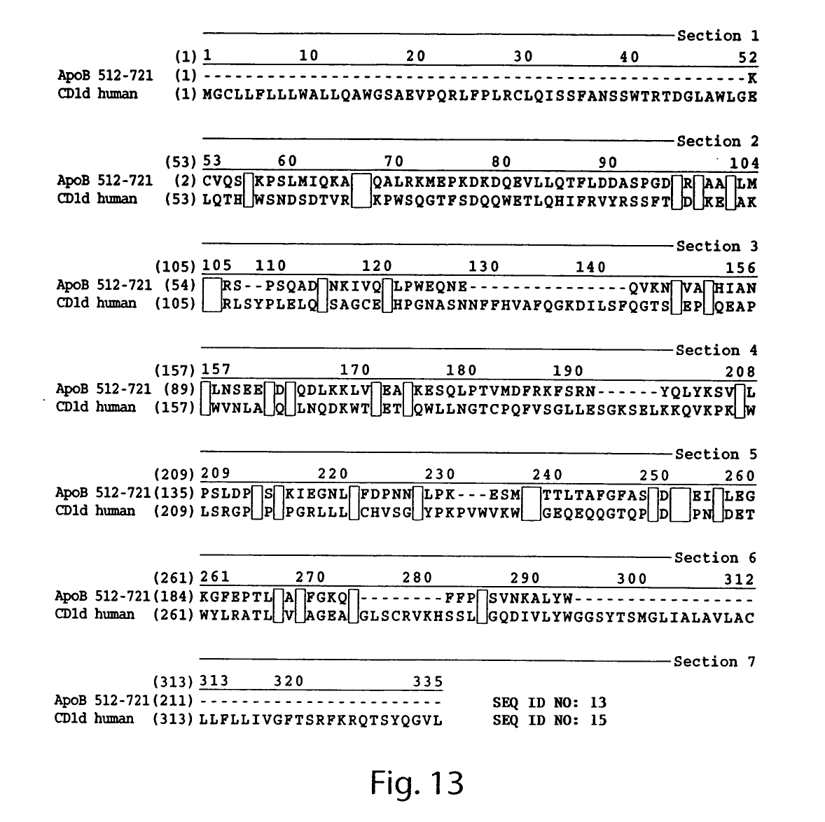

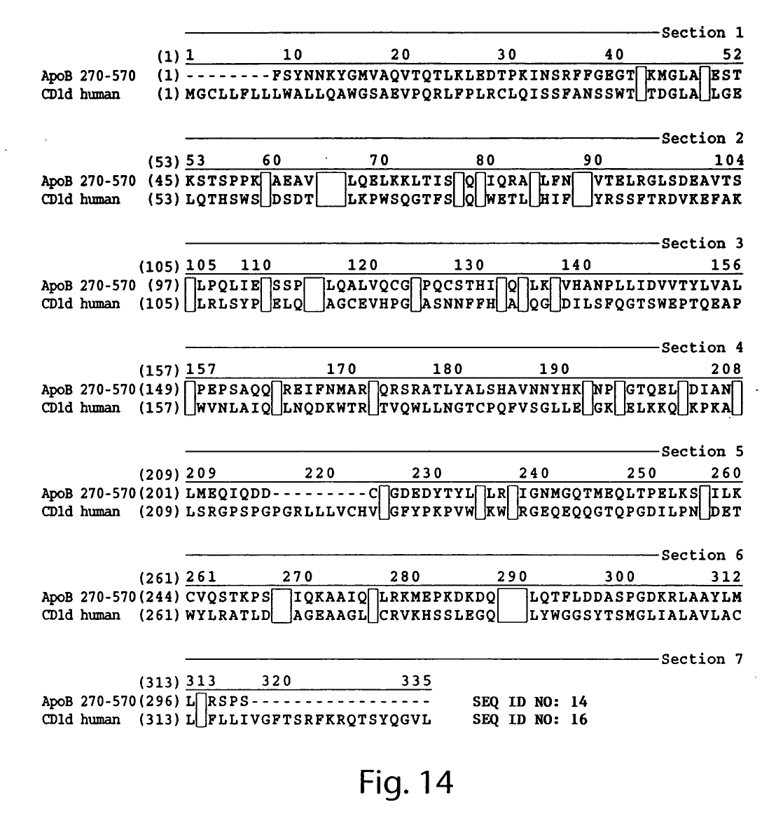

CD1 is a family of nonpolymorphic cell surface glycoproteins encoded outside the major histocompatibility complex (MHC) but with distant relationship to both MHC class I and class II molecules. The CD1 family is divided into two groups by sequence homology. Group I, consists of CD1a, -b, and -c isotopes and Group II contains CD1d and CD1e. The CD1 molecule comprises four domains; α1, α2, α3 and β2 m. MTP interacts with three region on apoB, one low affinity binding region corresponding to amino acid residues 1-269, and two high affinity binding regions corresponding to amino acids 270-570 and 512-721. Sequence aligment of these three MTP binding domains of apoB with CD1d identified regions throughout the CD1d molecule that align with these regions of apoB. (FIGS. 12-14) The analysis indicates that α1, α2 and α3 domains participate the lipid binding activity of CD1. Contacting these CD1 with a soluble compound that binds to or associates with one or more of these domains inhibits an CD1 activity, e.g. binding to MTP.

Hepatocytes from animals in which the Mttp gene has been conditionally deleted and IECs in which Mtpp gene products have been silenced by siRNA fail to activate iNKT cells. Conditional deletion of the Mttpp gene is associated with a redistribution of CD1d expression in hepatocytes and resistance of Mttp gene deleted mice to immunopathologies associated with iNKT mediated hepatitis and colitis. In vivo studies using a conditional MTP knockout mouse model demonstrated that deletion of the Mttp gene conferred protection of oxazolone-induced colitis. These studies indicate that MTP regulates the ability of CD1d-bearing cell types to exhibit CD1d-restricted antigen presentation. This result was surprising, as it would be expected that deletion of the Mttp gene would increase the sensitivity to tissue exposed to toxins.

These studies indicate that, similar to the relationship between MTP and apo-B²⁹, MTP is involved in the lipidation and functional maturation of CD1d, in addition to all CD1 molecules due to their structural and function similarity. This functional maturation of CD1 controlled by MTP includes the acquisition of glycolipid antigens involved in the normal function of CD1 in vivo and indicates that blockade of MTP function will be of therapeutic benefit in diseases mediated by CD1 and related pathways.

MTP Inhibitors

A MTP inhibitor is a compound that decreases expression or activity of MTP. A decrease in MTP expression or activity is defined by a reduction of a biological function of the MTP protein. A MTP biological function includes for example, the catalysis of lipid molecules between phospholipid membranes or the transfer of lipid from high density lipoproteins (HDL) to low density lipoproteins (LDL). MTP expression is measured by detecting a MTP transcript or protein. MTP inhibitors are known in the art or are identified using methods described herein. For example, a MTP inhibitor is identified by detecting a decrease the MTP-mediated transfer of lipids from HDL to LDL. Transfer of lipid is detected by methods known in the art such as nuclear magnetic resonance (NMR), electron spin resonance (ESR), radiolabeling or fluorescent labeling. For example, a decrease in transfer of lipid from HDL to LDL in the presence of the compound compared to the absence of the compound indicates a decrease in MTP activity. An MTP inhibitor is also identified by detecting the inhibition of the interaction between CD1 and MTP.

The MTP inhibitor is for example an antisense MTP nucleic acid, a MTP-specific short-interfering RNA, or a MTP-specific ribozyme. Exemplary nucleic acids and polypeptides encoding MTP include for example a human MTP available as GENBANK™ Accession No. NM000253 (SEQ ID NO:3 and SEQ ID NO: 4); Tables 1 and 2) or a murine MTP available as GENBANK™ Accession No. NM008642 (SEQ ID NO:5 and SEQ ID NO: 6; Tables 3 and 4). Start and stop codons are identified in bold in the nucleic acid sequences shown below.

TABLE 1
Human MTP Nucleic Acid (M subunit) (SEQ ID NO:3)

| | | | | | | |
|---|---|---|---|---|---|---|
| actccctcac | tggctgccat | tgaaagagtc | cacttctcag | tgactcctag | ctgggcactg | 61 |
| gatgcagttg | aggattgctg | gtcaatatga | ttcttcttgc | tgtgcttttt | ctctgcttca | 121 |
| tttcctcata | ttcagcttct | gttaaaggtc | acacaactgg | tctctcatta | aataatgacc | 181 |
| ggctgtacaa | gctcacgtac | tccactgaag | ttcttcttga | tcggggcaaa | ggaaaactgc | 241 |
| aagacagcgt | gggctaccgc | atttcctcca | acgtggatgt | ggccttacta | tggaggaatc | 301 |
| ctgatggtga | tgatgaccag | ttgatccaaa | taacgatgaa | ggatgtaaat | gttgaaaatg | 361 |
| tgaatcagca | gagaggagag | aagagcatct | tcaaaggaaa | aagcccatct | aaaataatgg | 421 |
| gaaaggaaaa | cttggaagct | ctgcaaagac | ctacgctcct | tcatctaatc | catggaaagg | 481 |
| tcaaagagtt | ctactcatat | caaaatgagg | cagtggccat | agaaaatatc | aagagaggtc | 541 |
| tggctagcct | atttcagaca | cagttaagct | ctggaaccac | caatgaggta | gatatctctg | 601 |
| gaaattgtaa | agtgacctac | caggctcatc | aagacaaagt | gatcaaaatt | aaggccttgg | 661 |
| attcatgcaa | aatagcgagg | tctggattta | cgaccccaaa | tcaggtcttg | ggtgtcagtt | 721 |
| caaaagctac | atctgtcacc | acctataaga | tagaagacag | ctttgttata | gctgtgcttg | 781 |
| ctgaagaaac | acacaatttt | ggactgaatt | tcctacaaac | cattaagggg | aaaatagtat | 841 |
| cgaagcagaa | attagagctg | aagacaaccg | aagcaggccc | aagattgatg | tctggaaagc | 901 |
| aggctgcagc | cataatcaaa | gcagttgatt | caaagtacac | ggccattccc | attgtggggc | 961 |
| aggtcttcca | gagccactgt | aaaggatgtc | cttctctctc | ggagctctgg | cggtccacca | 1021 |
| ggaaatacct | gcagcctgac | aacctttcca | aggctgaggc | tgtcagaaac | ttcctggcct | 1081 |
| tcattcagca | cctcaggact | gcgaagaaag | aagagatcct | tcaaatacta | aagatggaaa | 1141 |
| ataaggaagt | attacctcag | ctggtggatg | ctgtcacctc | tgctcagacc | tcagactcat | 1201 |
| tagaagccat | tttggacttt | ttggatttca | aaagtgacag | cagcattatc | ctccaggaga | 1261 |
| ggtttctcta | tgcctgtgga | tttgcttctc | atcccaatga | agaactcctg | agagccctca | 1321 |
| ttagtaagtt | caaaggttct | attggtagca | gtgacatcag | agaaactgtt | atgatcatca | 1381 |
| ctgggacact | tgtcagaaag | ttgtgtcaga | atgaaggctg | caaactcaaa | gcagtagtgg | 1441 |
| aagctaagaa | gttaatcctg | ggaggacttg | aaaaagcaga | gaaaaaagag | gacaccagga | 1501 |
| tgtatctgct | ggctttgaag | aatgccctgc | ttccagaagg | catcccaagt | cttctgaagt | 1561 |
| atgcagaagc | aggagaaggg | cccatcagcc | acctggctac | cactgctctc | cagagatatg | 1621 |
| atctcccttt | cataactgat | gaggtgaaga | agaccttaaa | cagaatatac | caccaaaacc | 1681 |
| gtaaagttca | tgaaaagact | gtgcgcactg | ctgcagctgc | tatcatttta | aataacaatc | 1741 |
| catcctacat | ggacgtcaag | aacatcctgc | tgtctattgg | ggagcttccc | caagaaatga | 1801 |
| ataaatacat | gctcgccatt | gttcaagaca | tcctacgttt | ggaaatgcct | gcaagcaaaa | 1861 |
| ttgtccgtcg | agttctgaag | gaaatggtcg | ctcacaatta | tgaccgtttc | tccaggagtg | 1921 |
| gatcttcttc | tgcctacact | ggctacatag | aacgtagtcc | ccgttcggca | tctacttaca | 1981 |
| gcctagacat | tctctactcg | ggttctggca | ttctaaggag | aagtaacctg | aacatctttc | 2041 |
| agtacattgg | gaaggctggt | cttcacggta | gccaggtggt | tattgaagcc | caaggactgg | 2101 |
| aagccttaat | cgcagccacc | cctgacgagg | gggaggagaa | ccttgactcc | tatgctggta | 2161 |
| tgtcagccat | cctctttgat | gttcagctca | gacctgtcac | ctttttcaac | ggatacagtg | 2221 |
| atttgatgtc | caaaatgctg | tcagcatctg | gcgaccctat | cagtgtggtg | aaaggactta | 2281 |
| ttctgctaat | agatcattct | caggaacttc | agttacaatc | tggactaaaa | gccaatatag | 2341 |
| aggtccaggg | tggtctagct | attgatattt | caggtgcaat | ggagtttagc | ttgtggtatc | 2401 |
| gtgagtctaa | aacccgagtg | aaaaataggg | tgactgtggt | aataaccact | gacatcacag | 2461 |
| tggactcctc | ttttgtgaaa | gctggcctgg | aaaccagtac | agaaacagaa | gcaggcttgg | 2521 |
| agtttatctc | cacagtgcag | ttttctcagt | acccattctt | agtttgcatg | cagatggaca | 2581 |
| aggatgaagc | tccattcagg | caatttgaga | aaaagtacga | aaggctgtcc | acaggcagag | 2641 |
| gttatgtctc | tcagaaaaga | aaagaaagcg | tattagcagg | atgtgaattc | ccgctccatc | 2701 |
| aagagaactc | agagatgtgc | aaagtggtgt | ttgcccctca | gccggatagt | acttccagcg | 2761 |
| gatggttttg | aaactgacct | gtgatatttt | acttgaattt | gtctccccga | aagggacaca | 2821 |
| atgtggcatg | actaagtact | tgctctctga | gagcacagcg | tttacatatt | tacctgtatt | 2881 |
| taagattttt | gtaaaaagct | acaaaaaact | gcagtttgat | caaatttggg | tatatgcagt | 2941 |
| atgctaccca | cagcgtcatt | ttgaatcatc | atgtgacgct | ttcaacaacg | ttcttagttt | 3001 |
| acttatacct | ctctcaaatc | tcatttggta | cagtcagaat | agttattctc | taagaggaaa | 3061 |
| ctagtgtttg | ttaaaaacaa | aaataaaaac | aaaaccacac | aaggagaacc | caattttgtt | 3121 |
| tcaacaattt | ttgatcaatg | tatatgaagc | tcttgatagg | acttccttaa | gcatgacggg | 3181 |
| aaaaccaaac | acgttcccta | atcaggaaaa | aaaaaaaaaa | aaaaaagtaa | gacacaaaca | 3241 |
| aaccattttt | ttctcttttt | ttggagttgg | gggcccaggg | agaagggaca | aggcttttaa | 3301 |
| aagacttgtt | agccaacttc | aagaattaat | atttatgtct | ctgttattgt | tagttttaag | 3361 |
| ccttaaggta | gaaggcacat | agaaataaca | tc | | | |

TABLE 2
Human MTP Amino Acid Sequence (SEQ ID NO:4)

MILLAVLFLCFISSYSASVKGHTTGLSLNNDRLYKLTYSTEVLL
DRGKGKLQDSVGYRISSNVDVALLWRNPDGDDDQLIQITMKDVNVENVNQQRGEKSIF
KGKSPSKIMGKENLEALQRPTLLHLIHGKVKEFYSYQNEAVAIENIKRGLASLFQTQL
SSGTTNEVDISGNCKVTYQAHQDKVIKIKALDSCKIARSGFTTPNQVLGVSSKATSVT
TYKIEDSFVIAVLAEETHNFGLNFLQTIKGKIVSKQKLELKTTEAGPRLMSGKQAAAI
IKAVDSKYTAIPIVGQVFQSHCKGCPSLSELWRSTRKYLQPDNLSKAEAVRNFLAFIQ
HLRTAKKEEILQILKMENKEVLPQLVDAVTSAQTSDSLEAILDFLDFKSDSSIILQER
FLYACGFASHPNEELLRALISKFKGSIGSSDIRETVMIITGTLVRKLCQNEGCKLKAV
VEAKKLILGGLEKAEKKEDTRMYLLALKNALLPEGIPSLLKYAEAGEGPISHLATTAL
QRYDLPFITDEVKKTLNRIYHQNRKVHEKTVRTAAAAIILNNNPSYMDVKNILLSIGE
LPQEMNKYMLAIVQDILRLEMPASKIVRRVLKEMVAHNYDRFSRSGSSSAYTGYIERS
PRSASTYSLDILYSGSGILRRSNLNIFQYIGKAGLHGSQVVIEAQGLEALIAATPDEG
EENLDSYAGMSAILFDVQLRPVTFFNGYSDLMSKMLSASGDPISVVKGLILLIDHSQE
LQLQSGLKANIEVQGGLAIDISGAMEFSLWYRESKTRVKNRVTVVITTDITVDSSFVK
AGLETSTETEAGLEFISTVQFSQYPFLVCMQMDKDEAPFRQFEKKYERLSTGRGYVSQ
KRKESVLAGCEFPLHQENSEMCKVVFAPQPDSTSSGWF

TABLE 3
Mouse MTP Nucleic Acid (SEQ ID NO:5)

| | | | | | | |
|---|---|---|---|---|---|---|
| ctggatgtgg | cagagggagc | cagcatgatc | ctcttggcag | tgctttttct | ctgcttcttc | 61 |
| tcctcctact | ctgcttccgt | taaaggtcac | acaactggcc | tctcattaaa | taatgagcgg | 121 |
| ctatacaagc | tcacgtactc | cactgaagtg | tttcttgatg | ggggcaaagg | aaaaccgcaa | 181 |
| gacagcgtgg | gctacaaaat | ctcatctgat | gtggacgttg | tgttactgtg | gaggaatcct | 241 |
| gatggtgatg | atgatcaagt | gatccaagtc | acgataacag | ctgttaacgt | tgaaaatgcg | 301 |
| ggtcaacaga | gaggcgagaa | gagcatcttc | cagggcaaaa | gtacacctaa | gatcataggg | 361 |
| aaggacaacc | tggaggctct | gcagagaccc | atgcttcttc | atctggtccg | ggggaaggtc | 421 |
| aaggagttct | actcctatga | aaacgagcca | gtgggcatag | aaaatctcaa | gagaggcttg | 481 |
| gctagcttat | tccagatgca | gctaagctct | ggaactacca | acgaggtaga | tatctctggg | 541 |
| gattgtaaag | tgacctacca | ggcccaacaa | gacaaagtgg | tcaaaattaa | ggctctggat | 601 |
| acatgcaaaa | ttgagcggtc | tggatttaca | acggcaaacc | aggtgctggg | cgtcagttca | 661 |
| aaagccacat | ctgtcactac | ctacaagata | gaggacagct | ttgtcaccgc | tgtgcttgca | 721 |
| gaagagacca | gggcttttgc | cttgaacttc | caacaaacca | tagcaggaaa | aatagtgtca | 781 |
| aagcagaaat | tggagctgaa | gacaactgaa | gccggcccaa | ggatgatccc | cgggaagcaa | 841 |
| gtggcaggtg | taattaaagc | agttgattcc | aaatacaaag | ccattcccat | tgtgggacag | 901 |
| gtcctcgagc | gtgtctgcaa | aggatgccct | tctctggcgg | agcactggaa | gtccatcaga | 961 |
| aagaacctgg | agcctgaaaa | cctgtccaag | gccgaggctg | tccagagctt | cctggccttc | 1021 |
| atccagcacc | tccggacttc | gaggagagaa | gagatcctcc | agattctgaa | ggcagagaag | 1081 |
| aaagaagtgc | tccctcagct | ggtggatgcc | gtcacctctg | ctcagactcc | agactcgcta | 1141 |
| gaagccatcc | tggacttttt | ggatttcaaa | agtgacagca | gtatcatact | ccaggaaagg | 1201 |
| ttcctctatg | cctgtggctt | tgccacccac | cctgatgaag | aactcctacg | agccctcctt | 1261 |
| agtaagttca | aaggttcctt | tgcaagcaac | gacatcagag | agtcggttat | gatcatcatt | 1321 |
| ggagccctag | tcaggaagct | gtgtcagaat | gaaggctgca | agctcaaggc | agtggtggaa | 1381 |
| gctaagaagc | tgatcctggg | aggacttgaa | aaaccagaga | agaaagaaga | caccacaatg | 1441 |
| tacctgctgg | ccctgaagaa | tgccttgctt | cccgaaggca | tcccgctcct | tctgaagtat | 1501 |
| gctgaggctg | gagaagggcc | cgtcagccac | ctggccacca | ctgttctcca | gagatacgat | 1561 |
| gtctccttca | tcacagatga | ggtgaagaag | accttgaaca | ggatatacca | ccagaatcgt | 1621 |
| aaggttcatg | agaagacggt | gcgcacaact | gccgctgctg | tcatcttaaa | gaacccatcc | 1681 |
| tacatggatg | tgaagaacat | cctgctgtcc | attggggaac | tcccgaaaga | gatgaacaaa | 1741 |
| tacatgctca | ccgttgtgca | agacatcctg | cattttgaaa | tgcctgcaag | caaaatgatc | 1801 |
| cgtcgagttc | tcaaggagat | ggctgttcac | aattatgacc | gtttctccaa | gagtggatcc | 1861 |
| tcttctgcct | atactggcta | cgtagaacgt | agcccccgtg | cagcgtccac | atacagcctt | 1921 |
| gacatccttt | actctggctc | tggcattctg | aggagaagta | acctgaacat | cttccagtac | 1981 |
| atcaaaggaa | cagagcttca | tggtagtcag | gtggtgattg | aagcccaagg | gctggaaggc | 2041 |
| ttaattgcag | ccactcctga | tgaaggagag | gagaaccttg | actcttatgc | tggcatgtca | 2101 |
| gccatcctgt | ttgatgttca | gcttaggcct | gtcacatttt | ttaatggata | cagtgatttg | 2161 |
| atgtccaaaa | tgctgtcggc | atccggcgac | cctgtcagcg | tggtgaaagg | gcttattctg | 2221 |
| ttaatagacc | attctcagga | tattcagctg | caatctggac | taaaggccaa | tatggagatc | 2281 |
| cagggtggtc | tagctattga | tatttctggt | tcaatggaat | tcagtctgtg | gtatcgcgag | 2341 |
| tctaaaaccc | gagtgaaaaa | tcgggtggct | gtggtgataa | ccagcgacgt | cacagtggat | 2401 |
| gcctcttttg | tgaaagctgg | tctggaaagc | agagcggaga | cagaggctgg | gctggagttc | 2461 |
| atctccacag | tgcagttctc | acagtacccg | ttcttggtct | gcatgcagat | ggacaaggct | 2521 |
| gaagccccac | tcaggcaatt | cgagacaaag | tatgaaaggc | tatctacagg | caggggatat | 2581 |
| gtctctcgga | gaagaaaaga | gagcctagtg | gccggatgtg | aactccccct | ccatcaacag | 2641 |
| aactctgaga | tgtgcaacgt | ggtattccca | cctcagccag | aaagcgataa | ctccggtgga | 2701 |
| tggttttgat | tcccgtgggt | tcccttccac | cagaacgata | tgctatgacg | tgcctgaccc | 2761 |
| ttgctctctg | agagcacagt | gtttacatat | ttacctgtat | ttaagatgtt | tgtaaagagc | 2821 |
| agtggagaac | ttcagttgat | taaagttgaa | cctattcagg | agaagaccca | cagtgtcc | |

TABLE 4
Mouse MTP Amino Acid Sequence (SEQ ID NO:6)

MILLAVLFLCFFSSYSASVKGHTTGLSLNNERLYKLTYSTEVFL
DGGKGKPQDSVGYKISSDVDVVLLWRNPDGDDDQVIQVTITAVNVENAGQQRGEKSIF
QGKSTPKIIGKDNLEALQRPMLLHLVRGKVKEFYSYENEPVGIENLKRGLASLFQMQL
SSGTTNEVDISGDCKVTYQAQQDKVVKIKALDTCKIERSGFTTANQVLGVSSKATSVT
TYKIEDSFVTAVLAEETRAFALNFQQTIAGKIVSKQKLELKTTEAGPRMIPGKQVAGV
IKAVDSKYKAIPIVGQVLERVCKGCPSLAEHWKSIRKNLEPENLSKAEAVQSFLAFIQ
HLRTSRREEILQILKAEKKEVLPQLVDAVTSAQTPDSLEAILDFLDFKSDSSIILQER
FLYACGFATHPDEELLRALLSKFKGSFASNDIRESVMIIIGALVRKLCQNEGCKLKAV
VEAKKLILGGLEKPEKKEDTTMYLLALKNALLPEGIPLLLKYAEAGEGPVSHLATTVL
QRYDVSFITDEVKKTLNRIYHQNRKVHEKTVRTTAAAVILKNPSYMDVKNILLSIGEL
PKEMNKYMLTVVQDILHFEMPASKMIRRVLKEMAVHNYDRFSKSGSSSAYTGYVERSP
RAASTYSLDILYSGSGILRRSNLNIFQYIKGTELHGSQVVIEAQGLEGLIAATPDEGE
ENLDSYAGMSAILFDVQLRPVTFFNGYSDLMSKMLSASGDPVSVVKGLILLIDHSQDI
QLQSGLKANMEIQGGLAIDISGSMEFSLWYRESKTRVKNRVAVVITSDVTVDASFVKA
GLESRAETEAGLEFISTVQFSQYPFLVCMQMDKAEAPLRQFETKYERLSTGRGYVSRR
RKESLVAGCELPLHQQNSEMCNVVFPPQPESDNSGGWF

By the term “siRNA” is meant a double stranded RNA molecule which prevents translation of a target mRNA. Standard techniques of introducing siRNA into a cell are used, including those in which DNA is a template from which an siRNA RNA is transcribed. The siRNA includes a sense MTP nucleic acid sequence, an anti-sense MTP nucleic acid sequence or both. Optionally, the siRNA is constructed such that a single transcript has both the sense and complementary antisense sequences from the target gene, e.g., a hairpin.

Binding of the siRNA to an MTP transcript in the target cell results in a reduction in MTP production by the cell. The length of the oligonucleotide is at least 10 nucleotides and may be as long as the naturally-occurring MTP transcript. Preferably, the oligonucleotide is 19-25 nucleotides in length. Most preferably, the oligonucleotide is less than 75, 50 , 25 nucleotides in length. For example, the MTP siRNA includes the nucleotides at positions 480-580 of SEQ ID NO:4. MTP siRNA oligonucleotides which inhibit MTP expression in mammalian cells include oligonucleotides containing SEQ ID NO: 1 and 2.

Exemplary MTP inhibitors include protease inhibitors, carboline compounds and compounds falling within Formula I: 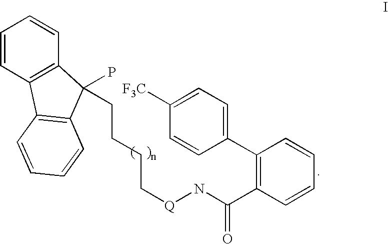
where n is zero or 1;
P is 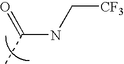
   or a 5- or 6-membered heterocycle selected from: 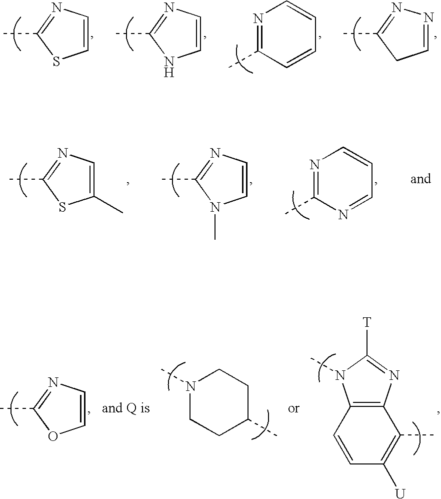
   where T and U are, independently, hydrogen or lower alkyl. (See, Robl, J. A. et al, Journal of Medicinal Chemistry (2001), 44(6): 851-6 and U.S. Pat. No. 6,281,228).

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. Unless the number of carbons is otherwise specified, “lower alkyl” as used herein means an alkyl group, as defined above, but having from one to ten carbons, more preferably from one to six carbon atoms in its backbone structure, and, if a cyclic alkyl, the term lower alkyl refers to those rings which have 5, 6, or 7 carbons in the ring structure.

Protease inhibitors inhibit secretion of apolipoprotein B through inhibition of microsomal triglyceride transfer-protein activity. (Liang, J. et al, Nature Medicine (2001), 7(12):1327-1331. Protease inhibitors include, for example, indinavir, ritonavir, nelfinavir and saquinavir. Other suitable protease inhibitors are known in the art.

Methods of Reducing Inflammation

Inflammation is inhibited by administering to tissue a MTP inhibitor. Tissues to be treated include a gastrointestinal tissue, e.g., intestinal tissue, a cardiac tissue, a pulmonary tissue, a dermal tissue, or a hepatic tissue. For example, the tissue is an epithelial tissue such as an intestinal epithelial tissue, pulmonary epithelial tissue, dermal tissue (i.e., skin), or liver epithelial tissue.

Inhibition of inflammation is characterized by a reduction of redness, pain and swelling of the treated tissue compared to a tissue that has not been contacted with a MTP inhibitor. Tissues are directly contacted with an inhibitor. Alternatively, the inhibitor is administered systemically. MTP inhibitors are administered in an amount sufficient to decrease (e.g., inhibit) inflammatory cytokine production. An inflammatory cytokine is a cytokine that modulates, e.g., induces or reduces an inflammatory response. An inflammatory response is evaluated by morphologically by observing tissue damage, localized redness, and swelling of the affected area. Alternatively, an inflammatory response is evaluated by measuring c-reactive protein, or IL-1 in the tissue or in the serum or plasma. An inflammatory cytokine is a proinflammatory cytokine. For example the inflammatory cytokine is, TNF alpha, interferon (e.g., alpha, beta or gamma), or interleukin (e.g., IL-1, IL-6, IL-0, IL-12, IL-14, IL-18). Cytokines are detected for example in the serum, plasma or the tissue. Cytokine production is measured by methods know in the art. For example, cytokine production is determined using an immunoassay specific for a Th1-specific or Th2-specific cytokine. A decrease in production of the cytokine in the presence of the compound compared to the level in the absence of the compound indicates a decrease in cytokine production. A decrease in white blood count also indicates a decrease in inflammation.

Efficaciousness of treatment is determined in association with any known method for diagnosing or treating the particular inflammatory disorder. Alleviation of one or more symptoms of the inflammatory disorder indicates that the compound confers a clinical benefit.

Alternatively, the cell is contacted with a MTP inhibitor in an amount sufficient to decrease T-cell activation, decrease antigen presentation or decrease expression of CD1d. T cells include T cytotoxic cells, T helper cells (e.g., Th1 and Th2) and natural killer T-Cells. The T-cell is a CD1d restricted T-cell. The T-cell express the natural killer receptor or an invariant T-cell receptor. T-cell activation is defined by an increase in calcium mediated intracellular cGMP or an increase in cell surface receptors for IL-2. For example, a decrease of T-cell activation is characterized by a decrease of calcium mediated intracellular cGMP and or IL-2 receptors in the presence of the compound compared to a the absence of the compound. Intracellular cGMP is measured for example, by a competitive immunoassay or scintillation proximity assay using commercially available test kits. Cell surface IL-2 receptors are measured for example, by determining binding to an IL-2 receptor antibody such as the PC61 antibody.

Antigen presentation the expression antigen on surface of a cell in a form recognizable by lymphocytes. Antigen presentation is determined by methods known in the art such as measuring IFN gamma production or IL-2 production. For example, an decrease of IFN gamma or IL-2 production in the presence of the MTP inhibitor as compared to the absence of the MTP inhibitor indicates a decrease in antigen presentation. IFN gamma or IL-2 production is measured for example, by binding to an ILN gamma or IL-2 antibody. A decrease in CD1d expression is defined by a decrease in cell surface expression. A decrease in CD1d cell surface expression is measured for example, by determining binding to a CD1d antibody.

Target cells includes those which express microsomal triglyceride transfer protein or which are induced to express MTP upon exposure to an inflammation trigger, e.g., infection, tissue damage, or exposure to an allergen. The target cell express CD1d. The cell is an immune cell such as an antigen presenting cell. The immune cell is for example as a B-cell, a monocyte, a macrophage, or a dendritic cell. The cell is a heart cell, a kidney cell, a brain cell, a yolk sac cell, a liver cell (i.e. hepatocyte), an epithelial cell or an intestinal cell. Preferably, the cell is an epithelial cell of the large or small intestine or the lung.

The methods are useful to alleviate the symptoms of a variety of inflammatory disorders. The inflammatory disorder is acute or chronic. Inflammatory disorders include cardiovascular inflammation, gastrointestinal inflammation, hepatic inflammatory disorders, pulmonary inflammation, kidney inflammation, ocular inflammation, pancreatic inflammation, genitourinary inflammation, autoimmune disease (e.g., diabetes, systemic lupus erythematosus, dermatomyositis, polymyositis, inflammatory neuropathies (Guillain Barré, inflammatory polyneuropathies), vasculitis (Wegener's granulomatosus, polyarteritis nodosa), polymyalgia rheumatica, temporal arteritis, Sjogren's syndrome, Bechet's disease, Churg-Strauss syndrome, Takayasu's arteritis), neuroinflammatory disorders (e.g., multiple sclerosis, allergy (e.g., allergic rhinitis/sinusitis, skin allergies and disorders (e.g., urticaria/hives, angioedema, atopic dermatitis, contact dermatitis, psoriasis), food allergies, drug allergies, insect allergies, mastocytosis), skeletal inflammation (e.g., arthritis, osteoarthritis, rheumatoid arthritis, spondyloarthropathies), infection ( e.g., bacterial or viral infections that depend on CD1d presentation such as Borrelia burgdorferi, Cryptococcus neoformans, Plasmodium falciparum, Trypanosoma cruzi, Leishmania major or viral hepatitis); oral inflammatory disorders ( i.e., perodontis, gingivitis or somatitis); and transplantation (e.g., allograft or xenograft rejection or maternal-fetal tolerance).

The methods described herein lead to a reduction in the severity or the alleviation of one or more symptoms of an inflammatory disorder such as those described herein. Inflammatory disorders are diagnosed and or monitored, typically by a physician using standard methodologies

Gastrointestinal Inflammatory Disorders

Gastrointestinal inflammatory disorders include for example, inflammatory bowel disease, Crohn's Disease, colitis (i.e., ulcerative, ileitis or proctitis).

Ulcerative colitis is an inflammatory bowel disease that causes inflammation and sores, called ulcers, in the top layers of the lining of the large intestine. The inflammation usually occurs in the rectum and lower part of the colon, but it can affect the entire colon. Ulcerative colitis rarely affects the small intestine except for the lower section, called the ileum. Ulcerative colitis occurs most often in people ages 15 to 40, although children and older people develop the disease. Ulcerative colitis affects men and women equally and appears to run in families. Crohn's Disease causes inflammation deeper within the intestinal wall. Crohn's disease usually occurs in the small intestine, but it can also occur in the mouth, esophagus, stomach, duodenum, large intestine, appendix, and anus.

Symptoms of gastrointestinal inflammatory disorder are abdominal pain and bloody diarrhea. Other symptoms include fatigue, weight loss, loss of appetite, rectal bleeding and loss of body fluids and nutrients. Gastrointestinal inflammation can also cause problems such as arthritis, inflammation of the eye, liver disease (fatty liver, hepatitis, cirrhosis, and primary sclerosing cholangitis), osteoporosis, skin rashes, anemia, and kidney stones.

Gastrointestinal inflammation is diagnosed using a blood tests to check for anemia, which can indicate bleeding in the colon or rectum. In addition, a stool sample, can be taken to determine is there is bleeding or infection in the colon or rectum. Alternatively, a colonoscopy is performed to detect inflammation, bleeding, or ulcers on the colon wall.

Hepatic Inflammatory Disorders

Hepatic inflammatory disorders include for example, hepatitis such viral hepatitis, bacterial hepatitis, autoimmune hepatitis, drug induced hepatitis or alcoholic hepatitis. The incidence and severity of hepatitis vary depending on many factors, including the cause of the liver damage and any underlying illnesses in a patient. Common risk factors include intravenous drug use, Tylenol overdose (the dose needed to cause damage is quite close to the effective dose so be sure to be careful to take Tylenol only as directed), risky sexual behaviors, ingestion of contaminated foods, and alcohol use.

Symptoms of hepatitis include dark urine, loss of appetite fatigue, jaundice, abdominal pain, black stool. Hepatitis is diagnosed by physical exam, liver function test, autoimmune marker and serology.

Pulmonary Inflammatory Disorders

Pulmonary inflammatory disorders include for example, sinusitis acute respiratory distress syndrome, asthma, bronchopulmonary dysplasia (BPD), emphysema, interstitial lung diseases, lung injury, and pulmonary hypertension.

Asthma is a chronic lung condition that can develop at any age. It is most common in childhood and occurs in approximately 7-10% of the pediatric population. Asthma affects twice as many boys as girls in childhood; more girls than boys develop asthma as teenagers, and in adulthood, the ratio becomes 1:1 males to females. Symptoms of asthma include shortness of breath, wheezing, constriction of the chest muscles, coughing, sputum production, excess rapid breathing/gasping, rapid heart rate and exhaustion. Asthma is diagnosed by physical examination, i.e. listening to the lungs with a stethoscope; examination of nasal passages, chest x-ray, blood tests or spirometry.

Cardiac Disorders

Cardiac inflammatory disorders include for example pericarditis, endocarditis, mycocarditis and atherosclerosis. Cardiac inflammation also includes an inflammation that results from an acute cardiac event such as a myocardial infarction. Cardiac inflammation is distinguished from other cardiac disorders in that inflammation is typically acute while other disorder such atherosclerosis are chronic. Atherosclerosis results in the build up of deposits of fatty substances, cholesterol, cellular waste products, calcium and in the inner lining of an artery (i.e., plaque) and has a significant inflammatory component. In contrast, cardiac inflammation affects the muscle tissue of the heart.

Pericarditis, is inflammation of the pericardium and is characterized by chest pain. Patients who have suffered a myocardial infarction often develop pericarditis over subsequent days or weeks. Pericarditis is diagnosed by elevated ST segments on an electrocardiogram.

Endocarditis is the inflammation of the endocardium and causes a wide variety of symptoms, particularly in the earlier stages of infection. Symptoms include fevers, chills, fatigue, weight loss, muscle aches, and sweating. Endocarditis is diagnoses by the presence of a heart murmur or an echocardiogram.

Myocarditis is the inflammation of the heart muscle. The symptoms of myocarditis include fever, chest pain, abnormal heat beats, fatique and shortness of breath. Myocarditis is typically diagnosed by a endomyocardial biopsy.

Therapeutic Administration

The invention includes administering to a subject a composition comprising a compound that decreases MTP expression or activity (referred to herein as an “MTP inhibitor” or “therapeutic compound”).

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other anti-inflammatory agents or therapeutic agents for treating, preventing or alleviating a symptom of a particular inflammatory disorder. A therapeutic regimen is carried out by identifying a mammal, e.g., a human patient suffering from (or at risk of developing) an inflammatory disorder, using standard methods.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The compound is administered prophylactically, or after the detection of an inflammatory event such as an asthma attack or an allergic reaction. The compound is optionally formulated as a component of a cocktail of therapeutic drugs to treat inflammatory disorders. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, MTP inhibitor is formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, a conventional filler, and a tableting agent. Other formulations include an ointment, suppository, paste, spray, patch, cream, gel, resorbable sponge, or foam. Such formulations are produced using methods well known in the art.

MTP inhibitor compounds are effective upon direct contact of the compound with the affected tissue. Accordingly, the compound is administered topically. For example, to treat contact dermatitis the compound is applied to the area of skin affected. Alternatively, MTP inhibitors are administered systemically. Additionally, compounds are administered by implanting (either directly into an organ such as the intestine, or liver or subcutaneously) a solid or resorbable matrix which slowly releases the compound into adjacent and surrounding tissues of the subject.

For example, for the treatment of gastrointestinal inflammatory disorders, the compound is systemically administered or locally administered directly into gastric tissue. The systemic administration compound is administered intravenously, rectally or orally. For local administration, a compound-impregnated wafer or resorbable sponge is placed in direct contact with gastric tissue. The compound or mixture of compounds is slowly released in vivo by diffusion of the drug from the wafer and erosion of the polymer matrix.

Inflammation of the liver (i.e., hepatitis) is treated for example by infusing into the liver vasculature a solution containing the compound. Intraperitoneal infusion or lavage is useful to reduce generalized intraperitioneal inflammation of prevent inflammation following a surgical event.

For the treatment of neurological inflammation the compound is administered intravenously or intrathecally (i.e., by direct infusion into the cerebrospinal fluid). For local administration, a compound-impregnated wafer or resorbable sponge is placed in direct contact with CNS tissue. The compound or mixture of compounds is slowly released in vivo by diffusion of the drug from the wafer and erosion of the polymer matrix. Alternatively, the compound is infused into the brain or cerebrospinal fluid using known methods. For example, a burr hole ring with a catheter for use as an injection port is positioned to engage the skull at a burr hole drilled into the skull. A fluid reservoir connected to the catheter is accessed by a needle or stylet inserted through a septum positioned over the top of the burr hole ring. A catheter assembly (e.g., an assembly described in U.S. Pat. No. 5,954,687) provides a fluid flow path suitable for the transfer of fluids to or from selected location at, near or within the brain to allow administration of the drug over a period of time.

For treatment of cardiac inflammation, the compound is delivered for example to the cardiac tissue (i.e., myocardium, pericardium, or endocardium) by direct intracoronary injection through the chest wall or using standard percutaneous catheter based methods under fluoroscopic guidance for direct injection into tissue such as the myocardium or infusion of an inhibitor from a stent or catheter which is inserted into a bodily lumen. Any variety of coronary catheter, or a perfusion catheter, is used to administer the compound. Alternatively, the compound is coated or impregnated on a stent that is placed in a coronary vessel.

Pulmonary inflammation is treated for example by administering the compound by inhalation. The compounds are delivered in the form of an aerosol spray from pressured container or dispenser which contains a suitable propellant, e.g., a gas such as carbon dioxide, or a nebulizer.

Evaluation of Anti-inflammatory Activity

Anti-inflammatory activity of a compound is identified by incubating cell with a compound and measuring inflammatory activity. A decrease in inflammation in the presence of the compound compared to the level in the absence of the compound indicates that the compound inhibits inflammation. Inflammatory activity is measured by detecting the production of cytokines such as IL-1. An increase in production of IL-1 in the presence of the compound compared to the amount detected in the absence of the compound indicates that the compound inhibits inflammation. A decrease in production of IL-1 in the presence of the compound compared to the amount detected in the absence of the compound indicates that the compound promotes inflammation. Inflammatory activity is also measured by detecting the amount of c-reactive protein or determining the erythrocyte sedimentation rate (ESR).

The following methods and reagents were used to generate the data described herein

Cells

The mouse (MODE-K) small IEC line has been previously described (van de Waal, Y. et al. Gastroenterology. 124, 1420-1431, (2003)). The murine Vα14/Jα281 invariant TCR-positive T cell hybridoma, DN32.D3, was kindly provided by Dr. Albert Bendelac (University of Chicago, Chicago, Ill.) (Bendelac, A. et al. Science. 268, 863-69 (1995)). DN32.D3 and MODE-K cells or primary murine hepatocytes were cultured in Dulbecco's modification of Eagle's medium (DMEM) (Gibco™ Invitrogen, Carlsbad, Calif.) supplemented with 10% FBS (Sigma-Aldrich, St. Louis, Mo.), 100 U/ml penicillin, 100 μg/ml streptomycin (Bio Whittaker, Walkersville, Md.), 10 mM HEPES (Gibco Invitrogen), and 1% non-essential amino acid (Mediatech, Herndon, Va.), as complete DMEM.

Animals Conditional MTPp gene deficient mice (MTP^{flox/flox}) were bred with Mx1 promoter-driven Cre recombinase transgenic mice as previously described (Raabe, M. et al. J. Clin. Invest. 103, 1287-1298 (1999)). Wild type (WT) C57BL/6J (B6) mice were purchased from Charles River Breeding Laboratories (Wilmington, Mass.). Mice were maintained under specific pathogen free conditions at the Animal Facilities of Harvard Medical School, Boston, Mass. All animal experimentations were performed in accordance with institutional guidelines and the Review Board of Harvard Medical School, which has granted permission for this study.

Assessment of Hepatic and Plasma Lipids after MTPp Deletion

Female 6-8 week-old of both MTP^{pflox/flox}Mx1Cre mice and WT B6 mice that had been injected intraperitoneally with either PBS as control or 500 μg polyinosinic-polycytidylic ribonucleic acid (pIpC, Sigma, St. Louis, Mo.) in PBS every other day for a total of four doses were sacrificed one or 10 days after the last pIpC injection at which time sera was obtained from peripheral blood. The serum was subjected to total cholesterol and triglyceride assays at the clinical laboratory of Brigham and Women's Hospital (Boston, Mass.).

Fresh liver samples that were collected from mice before pIpC injection or one day and 10 days after injection and immediately placed in OCT compound (Sakura Finetek USA, Inc., Torrance, Calif.) on dry ice and 5 μm thick cryosections prepared. Sections were air dried and fixed in 10% formalin (Fisher, Pittsburgh, Pa.) for 30 min followed by administration of oil-red-O by a standard protocol. Briefly, samples were washed with distilled water and rinsed twice for 5 min in 100% propylene glycol (Fisher). The samples were then stained with 7 mg/ml oil-red-O (Sigma) dissolved in propylene glycol with agitation and subsequently soaked in 85% (v/v with distilled water) propylene glycol for 3 min. After rinsing with distilled water, nuclear staining was performed with hematoxylin (VWR, West Chester, Pa.).

Cell Isolations and Antigen Presentation Assays

Fresh livers and colons were collected from MTP^{fox/flox}Mx1Cre mice treated with either PBS or pIpC. Livers were crushed on 70 μm cell strainers (BD Biosciences, San Jose, Calif.) and total hepatocytes were further fractionated on a 30% Percoll (Amersham Biosciences) gradient by centrifugation at 2000 rpm for 20 min. Primary hepatocytes were isolated from the surface fraction and antigen presentation assays performed as previously described²⁷. Briefly, 1×10⁵ primary hepatocytes in 100 μl DMEM were loaded overnight with either vehicle or 100 ng/ml αGalCer in flat bottom 96-well plates. The plates were subsequently washed twice with PBS preceding the addition of 5×10⁴ DN32.D3 cells. Culture supernatants were harvested after 24 h and subjected to determination of murine IL-2 production by ELISA (OptEIA, BD Pharmingen, San Diego, Calif.) according to the manufacturer's instructions.

Colonic specimens were washed with HBSS, cut in 3 mm pieces and incubated twice in HBSS containing 5 mM EDTA (Sigma) and 1 mM DTT (Sigma) in a shaking incubator at 37° C. for 20 min. Following the collection of supernatant, cells were further fractionated on a 30% Percoll gradient by centrifugation at 2000 rpm for 20 min. Primary colonic epithelial cells were isolated from the surface fraction.

The expression levels of MTP protein in primary hepatocytes and colonic epithelial cells were assessed by RT-PCR (see below) and Western blotting by a standard protocol. Briefly, 20 μg cell lysates were applied to 6% SDS-PAGE and then transferred to a nitrocellulose membrane. Following the blocking with 5% skim milk, the membrane was incubated with specific antibodies and the signals generated were detected by ECL Western blotting analysis system (Amersham Biosciences). The specific antibodies used included either anti-CD1d monoclonal antibody, 1B1 (BD Pharmingen), anti-MTP antibody (BD Biosciences), goat anti-mouse IgG2a-HRP (Southern Biotechnology Associates, Inc., Birmingham, Ala.), rat IgG2b antibody (BD Pharmingen) or mouse IgG2a (BD Pharmingen).

Reverse Transcriptase-Polymerase Chain Reaction Amplification (RT-PCR)

Total cellular RNA was extracted using Trizol (Invitrogen, Carlsbad, Calif.) following the manufacture's instructions. 100 ng RNA was subjected to reverse-transcription using the Advantage RT-for-PCR Kit (Clontech Laboratories, Palo Alto, Cailf.). The sense and antisense primers for murine MTPp were 5′-GGACTTTTTGGATTTCAAAAGTGAC-3′ and 5′-GGAGAAACGGTCATAATTGTG-3′ (SEQ ID NO:7). The conditions for the PCR were as follows: after initial denaturation at 95° C. for 5 min, the thermocycler (MJ Research, Watertown, Mass.) was programmed for 35 cycles: 1 min at 95° C., 1 min at 55° C. and 2 min at 72° C. The reaction was concluded with a final extension step at 72° C. for 7 min. 1 μl of each PCR reaction mixture was separated and visualized with 1.5% agarose gel electrophoresis containing 0.01% ethidium bromide. Optical density of the cDNA bands were determined by a computerized image-analysis system and normalized to the RT-PCR products of P-actin with the following primer pair: 5′-GTGGGCCGCTCTAGGCACCAA-3′ (SEQ ID NO:8) and 5′-CTCTTTGATGTCACGCACGATTTC-3′ (SEQ ID NO: 9).

Immunohistochemistry and Confocal Microscopy

MTP^{flox/flox}Mx1Cre mice that had been treated with either vehicle or pIpC were sacrificed and fresh liver samples immediately placed in OCT compound (Sakura Finetek USA) on dry ice and 5 μm cryosections prepared. Sections were air dried and fixed in cold acetone for 30 min. Samples were rehydrated in PBS for 5 min and blocked in PBS with 10 % goat serum (Zymed Laboratories, Inc South San Francisco, Calif.) for 20 min. The sections were incubated with rat anti-mouse CD1d monoclonal antibody, 1B1, or rat anti-mouse CEACAM1 antibody (clone AgB10) (kindly provided by Dr. Nicole Beauchemin, McGill University, Montreal, Canada), followed by Alexa⁴⁸⁸ conjugated goat anti-rat IgG secondary antibodies and rhodamine conjugated phalloidin (Molecular Probe, Eugene, Oreg.) for 30 min at RT. After washing three times in PBS for 5 min, nucleii were stained with TO-PRO-3 (Molecular probe) and tissues mounted and preserved with Prolong Antifade reagent (Molecular probe). All images were collected using a MRC1024 laser scanning confocal system (Bio-Rad Laboratories, Hercules, Calif.) using the same laser power, gain, and pinhole size for the respective channels.

αGalCer-Induced Hepatitis

αGalCer was kindly provided by Dr. Michael Brenner (Brigham and Women's Hospital, Boston, Mass.). To generate αGalCer-induced hepatitis, 6 week-old female WT B6 or MTP^{flox/flox}Mx1Cre mice were injected intraperitoneally with PBS or 500 μg pIpC at days 1, 3, 5 and 7 and injected intraperitoneally with 2 μg/mouse of αGalCer on either day 8 or day 18. Mice were sacrificed and sera from peripheral blood subjected to aminoleucine transferase (ALT) or aminoaspartate transferase (AST) assays. The kinetic quantitative determination of ALT in serum was performed by ALT/SGPT LIQUI-UV (Stanbio Laboratory, Boerne, Tex.) according to the manufacture's instructions. Fresh liver samples were collected for macroscopic and microscopic inspection. For microscopic examination, liver tissues were immediately fixed in 10% buffered formalin phosphate and embedded in paraffin, cut into sections and stained with hematoxylin-eosin.

Mttp Gene Silencing by Means of Small Interference RNA

To selectively silence MTPp gene expression in the murine IEC line, MODE-K, a specific small interference (si) RNA approach was developed. The siRNA duplexes were generated and consisted of a sense strand (5′-AAGCUCUGGAACUACCAACGAdTdT-3′ SEQ ID NO:1)) and an anti-sense strand (5′-UCGUGGUAGUUCCAGAGCUUdTdT-3′ SEQ ID NO:2) (Xeragon Inc. Germantown, Md.). 3 μg siRNA was used to transfect 5×10⁵ cells using the TransMessenger Transfect kit (Qiagen Inc., Valencia, Calif.) following the manufacturer's instructions. To confirm the post-transcriptional gene silencing effect of MTPp-specific siRNA, the transfected cells were harvested 48 h after transfection. Total cellular RNA was then extracted and subjected to RT-PCR analysis as described above.

Hapten-Induced Colitis

The hapten-induced colitis model using oxazolone (4-ethoxymethylene-2-phenyl-2-oxazolin-5-one) as the hapten was established in WT B6 or MTP^{flox/flox}Mx1Cre mice as previously described²⁸. Briefly, 6 week-old female mice were injected intraperitoneally with vehicle or 500 μg pIpC at days 1, 3, 5 and 7 followed by application of 200 μl of 3% oxazolone (Sigma-Aldrich, St. Louis, Mo.) in 100% ethanol on the abdomen at day 8. On day 14, mice were anesthetized with tribromo-ethanol (Sigma-Aldrich) and 150 μl of 1% oxazolone in 50% ethanol administered per rectum via a 3.5 F catheter. Wasting was monitored until day 18 at which time the mice were sacrificed and tissue samples collected for histological examination. Colonic tissue specimens for histological assessment of colitis were fixed in 10% buffered formalin phosphate and embedded in paraffin, cut into 5 micron thick sections and stained with hematoxylin-eosin. The stained paraffin sections were evaluated by for the following parameters of colitis on a scale of 0 to 3 according to known methods: hypervascularity, mononuclear cell infiltration, crypt hyperplasia, epithelial injury or ulceration, polymorphonuclear cell infiltration or crypt abscesses.

Statistical Analysis

Data are expressed as the mean±SEM and statistical significance determined by the Student's t-test. P values <0.05 were considered significant.

### EXAMPLE 1

### MTPp Gene Deletion Results in Redistribution of CD1d Expression in Hepatocytes

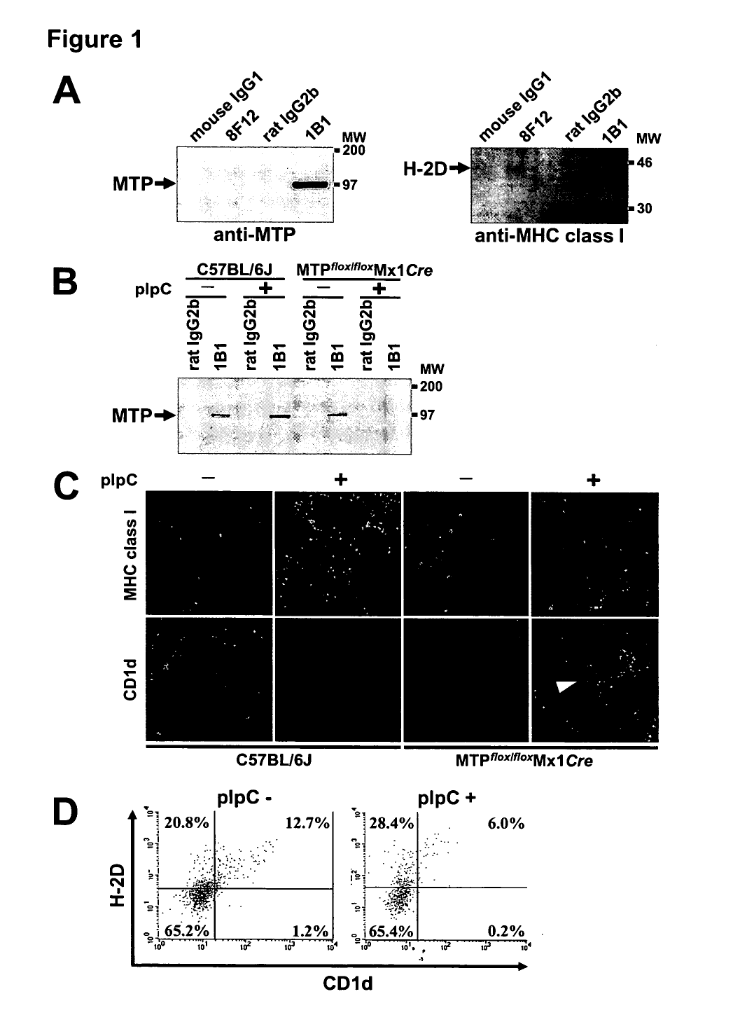

CD1d and MTP were found to co-associate with each other in hepatocytes. As shown in FIG. 1A, when protein lysates of hepatocytes from C57BL/6J mice were immunoprecipitated with a CD1d-specific monoclonal antibody and the immunoprecipitates resolved by SDS-PAGE followed by Western blotting, a specific band of 97-kDa consistent with MTP was detected.

To further substantiate a direct biochemical relationship between CD1d and MTP, the effects on CD1d expression and function was examined in a conditional MTP-deficient mouse model which contains a “floxed” MTPp gene (MTPp^{flox/flox}) that had been intercrossed with mice expressing Mx1 promotor controlled, Cre-recombinase (Raabe, M. et al. J. Clin. Invest. 103, 1287-1298 (1999); Bjorkegren, J., Beigneux, A., Bergo, M. O., Maher, J. J. & Young, S. G. J. Biol. Chem. 277, 5476-5483 (2002)). The Mx1 promotor is induced by interferon-inducing substances such as polyinosinic-polycytidylic acid (pIpC) (Raabe, M. et al. J. Clin. Invest. 103, 1287-1298 (1999); Bjorkegren, J., Beigneux, A., Bergo, M. O., Maher, J. J. & Young, S. G. J. Biol. Chem. 277, 5476-5483 (2002); Ralf, K., Schwenk, F., Aguet, M. & Rajewsky, K. Science. 269, 1427-1429 (1995)) such that treatment of MTPp^{flox/flox}Mx1Cre mice with pIpC leads to deletion of the MTPp gene (MTPp^{Δ/Δ}) primarily within the liver, spleen and intestine providing a degree of tissue specificity to the gene deletion²⁵. As previously reported^{22,24}, treatment of 6-10 week old MTPp^{flox/flox}Mx1Cre mice (or MTP^{flox/flox}Mx1Cre mice) with pIpC caused progressive hepatic steatosis (1 C) and decreases in serum triglyceride and cholesterol levels (Table 1) in association with decreased MTP mRNA levels as defined by RT-PCR (FIG. 1D) and protein levels (FIG. 1E) in liver and colon.

Given the detection of a biochemical association between CD1d and MTP (FIG. 1A), it was hypothesized that MTP-deficiency induced by Mx-1 regulated Cre activity would affect CD1d expression and/or function in the hepatocyte in vivo. Therefore, the expression and distribution of CD1d in the liver of MTP^{flox/flox}Mx1Cre mice before and after pIpC treatment was examined. Prior to pIpC treatment, CD1d was present diffusely throughout the hepatocyte with a membranous staining pattern as shown by the green fluorescence (FIG. 1B, panel a). In comparison, after pIpC treatment, CD1d in hepatocytes from MTP^{Δ/Δ} mice was noted to retreat into a prominent perinuclear staining pattern with reduced cell surface expression (FIG. 1B, panel b). In contrast, pIpC treatment of MTP^{flox/flox}Mx1Cre mice did not affect the distribution of either phalloidin staining of actin (FIG. 1B, panel b) or staining for carcinoembryonic antigen cell adhesion molecule 1 expression, a constitutive cell surface molecule on hepatocytes (FIG. 1B, panels c and d). These results indicate that the intracellular trafficking of CD1d is selectively influenced by the presence of MTP within hepatocytes in vivo.

TABLE 1
Characteristics of MTP^{flox/flox}Mx1Cre and WT C57BL/6J mice after pIpC
treatment.^{a}
1 day after treatment

| | MTP^{flox/flox}Mx1Cre (n = 9) | MTP^{flox/flox}Mx1Cre + pIpC (n = 9) | p value^{b} |
|---|---|---|---|
| Total S. Cholesterol^{c} | 144.0 ± 8.9 | 55.1 ± 9.7 | 0.02 |
| (mg/dl) | | | |
| Serum triglycerides | 62.6 ± 4.6 | 52.0 ± 7.5 | 0.25 |
| (mg/dl) | | | |
| Body weight (g) | 20.5 ± 0.6 | 21.8 ± 1.0 | 0.97 |
| | C57BL/6J (n = 10) | C57BL/6J + pIpC (n = 10) | p value |
| Total S. Cholesterol | 108.5 ± 12.0 | 96.2 ± 12.2 | 0.07 |
| Serum triglycerides | 112.1 ± 15.0 | 88.1 ± 6.6 | 0.91 |
| Body weight | 22.4 ± 0.6 | 21.5 ± 0.7 | 0.37 |

10 days after treatment

| | MTP^{flox/flox}Mx1Cre (n = 10) | MTP^{flox/flox}Mx1Cre + pIpC (n = 10) | p value |
|---|---|---|---|
| Total S. Cholesterol | 116.0 ± 8.6 | 49.2 ± 3.1 | 0.001 |
| Serum triglycerides | 63.2 ± 6.2 | 43.5 ± 7.3 | 0.02 |
| Body weight | 20.5 ± 0.6 | 21.8 ± 1.0 | 0.34 |
| | C57BL/6J (n = 8) | C57BL/6J + pIpC (n = 8) | p value |
| Total S. Cholesterol | 131.0 ± 9.5 | 127.0 ± 5.8 | 0.22 |
| Serum triglycerides | 131.5 ± 18.6 | 110.0 ± 11.2 | 0.94 |
| Body weight | 22.4 ± 0.6 | 20.7 ± 0.7 | 0.08 |

^{a}Female 6-8 week-old mice 1 day (A) or 10 days (B) after either vehicle or pIpC treatment were analyzed.
Data represent means ± SE.
^{b}P-values were calculated by two tailed unpaired t-test.
^{c}Total serum cholesterol.

### EXAMPLE 2

### Deletion of MTPp Gene Inhibits CD1d-Restricted Presentation by Hepatocytes

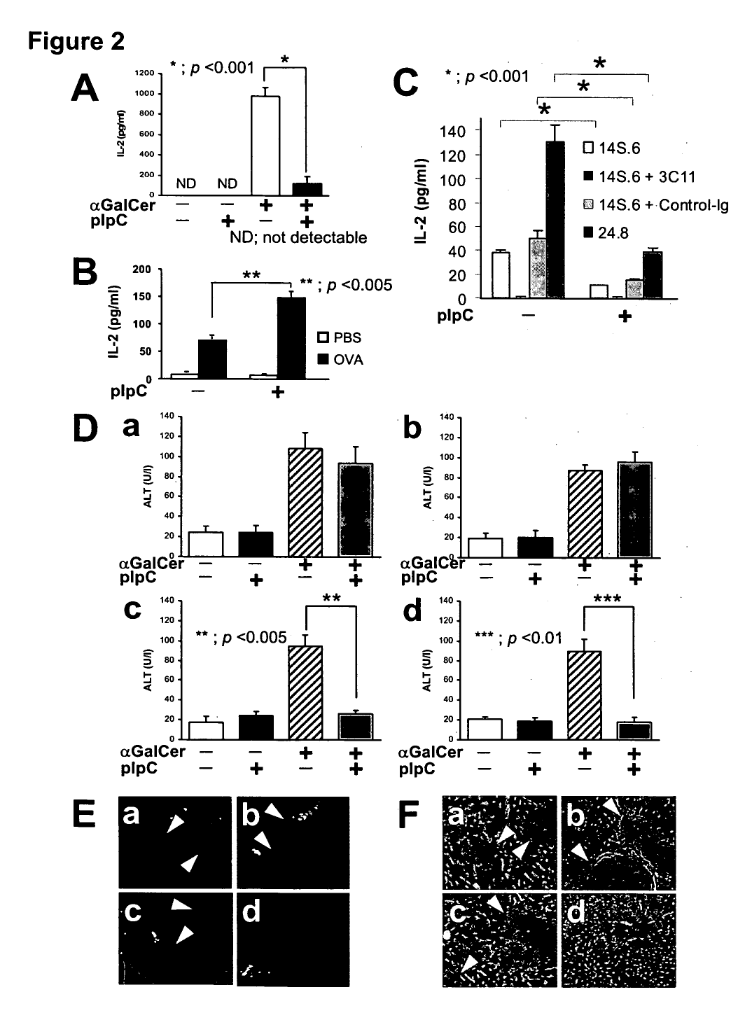

The effect of MTPp gene deletion upon the ability of CD1d expressed by hepatocytes to activate iNKT cells was determined. Hepatocytes obtained from either pIpC treated or untreated MTP^{flox/flox}Mx1Cre mice were therefore co-cultured with a mouse CD1d-restricted iNKT cell hybridoma, DN32.D3. As shown in FIG. 2A, hepatocytes from untreated MTP^{flox/flox}Mx1Cre mice stimulated the DN32.D3 cells to secrete interleukin-2 (IL-2) in the presence but not absence (vehicle control) of α-galactosylceramide (αGalCer). In contrast, hepatocytes from mice in which the MTPp gene had been deleted by pIpC treatment, stimulated little IL-2 production by the DN32.D3 cell line even in the presence of αGalCer (FIG. 2A), indicating a direct demonstration that MTP in hepatocytes is linked to the regulation of CD1d-restricted T cell responses by this cell type. Significantly, this effect was specific for CD1d since MHC class II-restricted activation of OVA-specific CD4⁺ T cells from OT-II transgenic mice by CD11c⁺ spleen cells from MTP^{flox/flox}Mx1Cre mice was unaffected by deletion of the MTPp gene.

### EXAMPLE 3

### Deletion of MTPp Gene Inhibits αGalCer-Induced Hepatitis

The clinical response to a hepatocyte mediated, CD1d-dependent response in vivo was evaluated (Osman, Y. et al. Eur. J. Immunol. 30, 1919-1928 (2000)). As would be expected, administration of αGalCer prior to pIpC treatment induced hepatitis in both wild-type (WT) C57BL/6J (B6) mice (FIG. 2B, panels a and b) and MTP^{flox/flox}Mx1Cre mice (FIG. 2B, panels c and d). The absence of inflammation with pIpC treatment alone is consistent with the lack of hepatic inflammation in either the absence per se of the MTPp gene (Raabe, M. et al. J. Clin. Invest. 103, 1287-1298 (1999)). or the presence of Mx1 activity induced by interferons (FIG. 2B, panels a-d) (Ralf, K., Schwenk, F., Aguet, M. & Rajewsky, K. Science. 269, 1427-1429 (1995)). In contrast to what would be predicted by the presence of hepatic steatosis induced by the lack of MTP function (Bjorkegren, J., Beigneux, A., Bergo, M. O., Maher, J. J. & Young, S. G. J. Biol. Chem. 277, 5476-5483 (2002)), MTPp^{Δ/Δ} mice were nearly completely protected from the inflammatory effects of αGalCer administration at either one day (FIG. 2D, panel c) or 10 days (FIG. 2B, panel d) after completion of the pIpC treatment regimen. The latter was evident from the absence of elevations of transaminases (FIG. 2D, panels c and d), macroscopic liver necrosis (FIG. 2E, panel d) or microscopic evidence of hepatitis (FIG. 2F, panel d) in the MTP^{flox/flox}Mx1Cre mice that received αGalCer with prior pIpC treatment. In contrast, WT B6 mice that received αGalCer at both one day (FIG. 2D, panel a) and 10 days (FIG. 2D, panel b) after pIpC treatment exhibited severe hepatitis. These results indicate in the absence of MTP, the liver is protected from CD1d-restricted and iNKT cell-mediated hepatocyte injury.

Significantly, this effect was specific for CD1d since MHC class II-restricted activation of OVA-specific CD4⁺ T cells from OT-II transgenic mice by CD11c⁺ dendritic cells obtained from the liver of MTP^{flox/flox}Mx1Cre mice was unaffected by deletion of the Mttp gene and, in fact, was increased consistent with the effects of interferon-α induced by pIpC treatment (FIG. 2D). CD1d-restricted autoreactivity to hepatocytes was also defective in the absence of MTP and affected T cells bearing either an invariant (24.8) or noninvariant (14S.6) TCR-α chain (FIG. 2C).

### EXAMPLE 4

### Inhibition of the MTPp Gene Inhibits Cd1d-Restricted Antigen Presentation

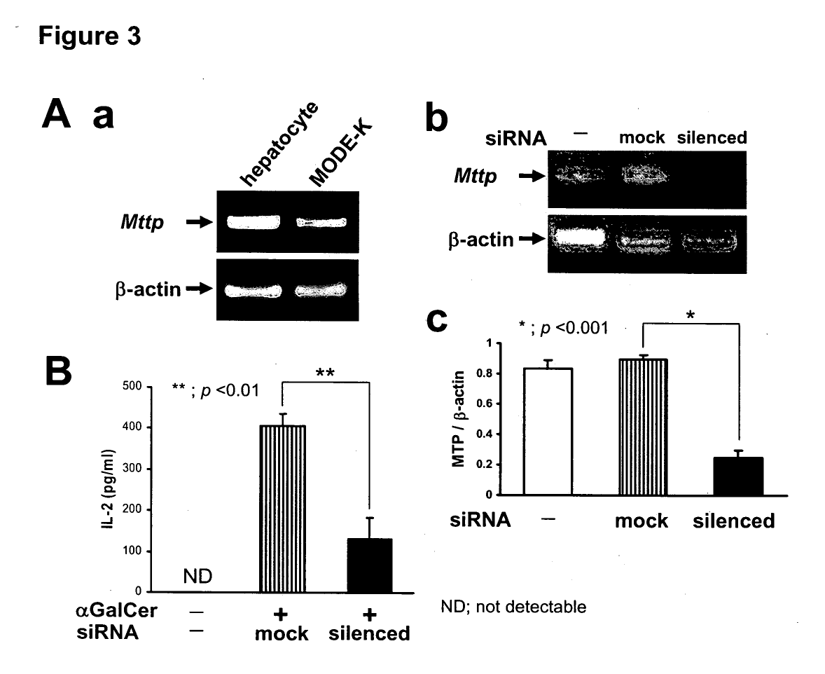

The relationship between MTP and CD1d in large intestinal epithelial cells (IECs) was determined. As predicted (Gordon, D. A., Wetterau, J. R. & Gregg, R. E. Trends Cell. Biol. 5, 317-321 (1995)), MTP was expressed by a mouse IEC line, MODE-K (FIG. 3A, panel a), which is also known to functionally express CD1d (van de Waal, Y. et al. Gastroenterology. 124, 1420-1431, (2003)). Gene silencing of MTPp expression in MODE-K cells with siRNA oligomers specific for MTPp transcripts caused a significant reduction in MTPp mRNA levels in the MODE-K cell line (FIG. 3B, panels b and c). In the absence of an exogenous source of the glycolipid antigen αGalCer, the MODE-K cell line was incapable of stimulating IL-2 production by DN32.D3 cells (FIG. 3B). In contrast, in the presence of αGalCer, the MODE-K cell line stimulated significant IL-2 production by the DN32.D3 cell line (FIG. 3B). This production of IL-2 was inhibited by silencing MTPp gene products using siRNA-specific oligomers but not siRNA oligomers directed at an irrelevant gene target (FIG. 3B). These studies indicate that. MTP regulates the ability of IECs to exhibit CD1d-restricted antigen presentation.

### EXAMPLE 6

### Inhibition of Oxazolone Induced Colitis in Microsomal Triglyceride Transferase Deficient Mice

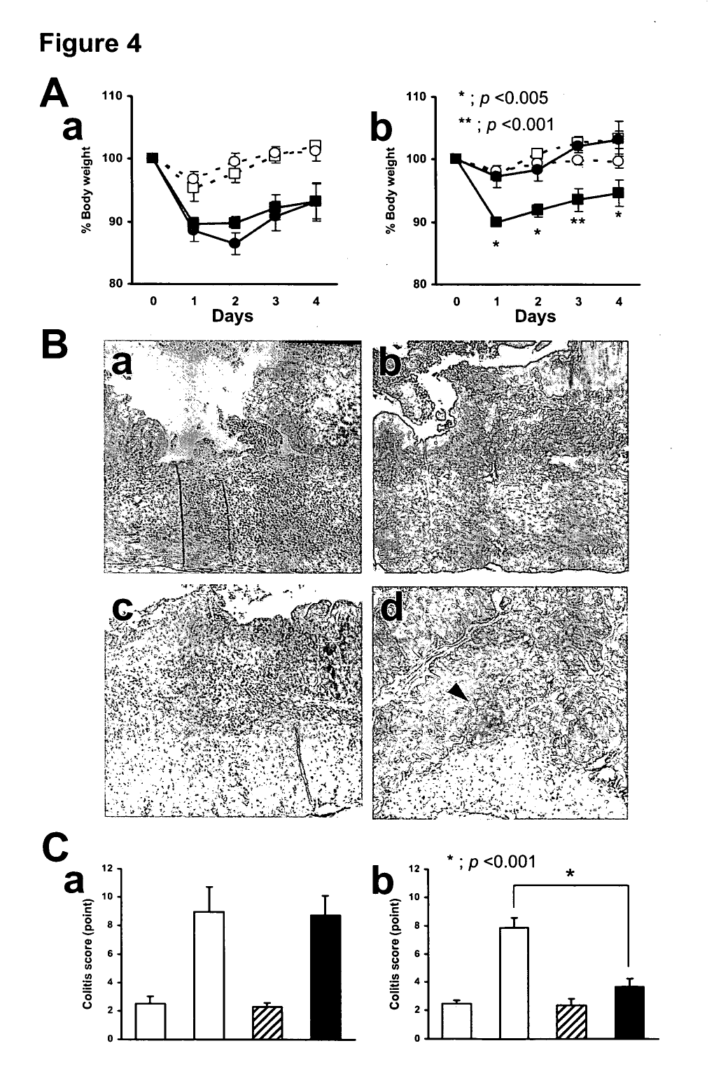

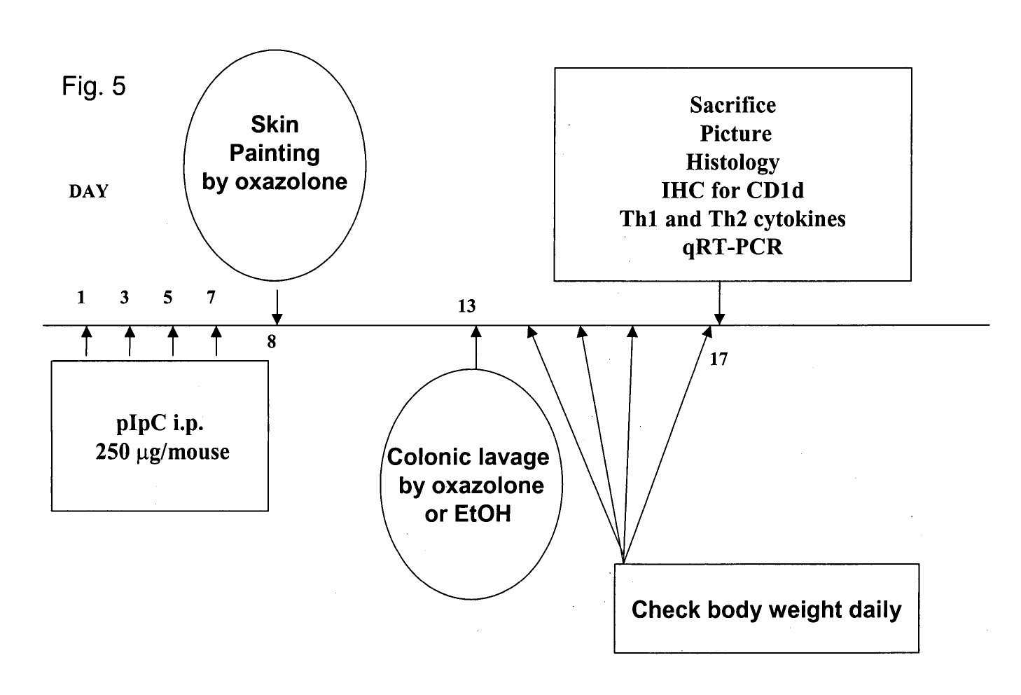

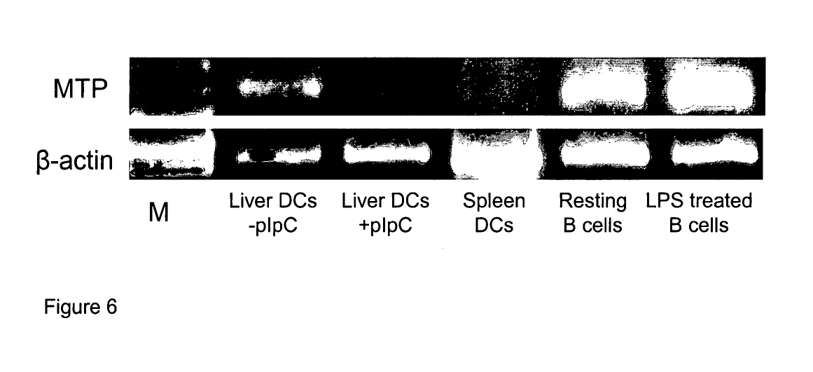

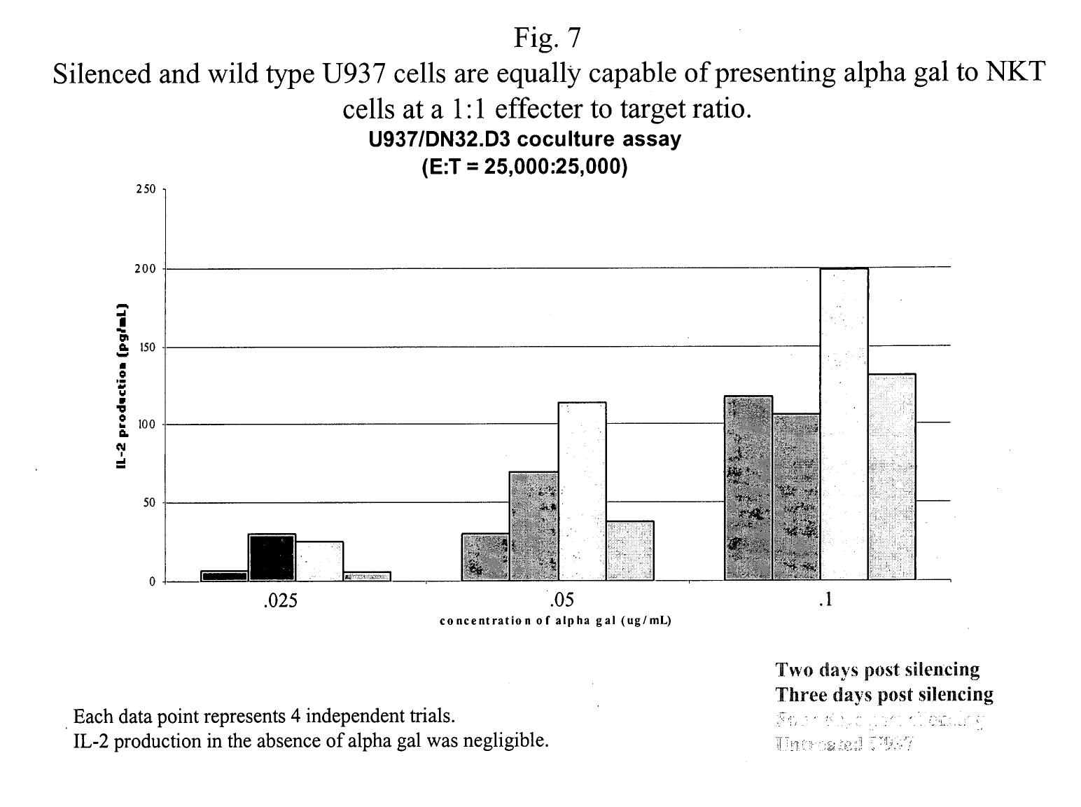

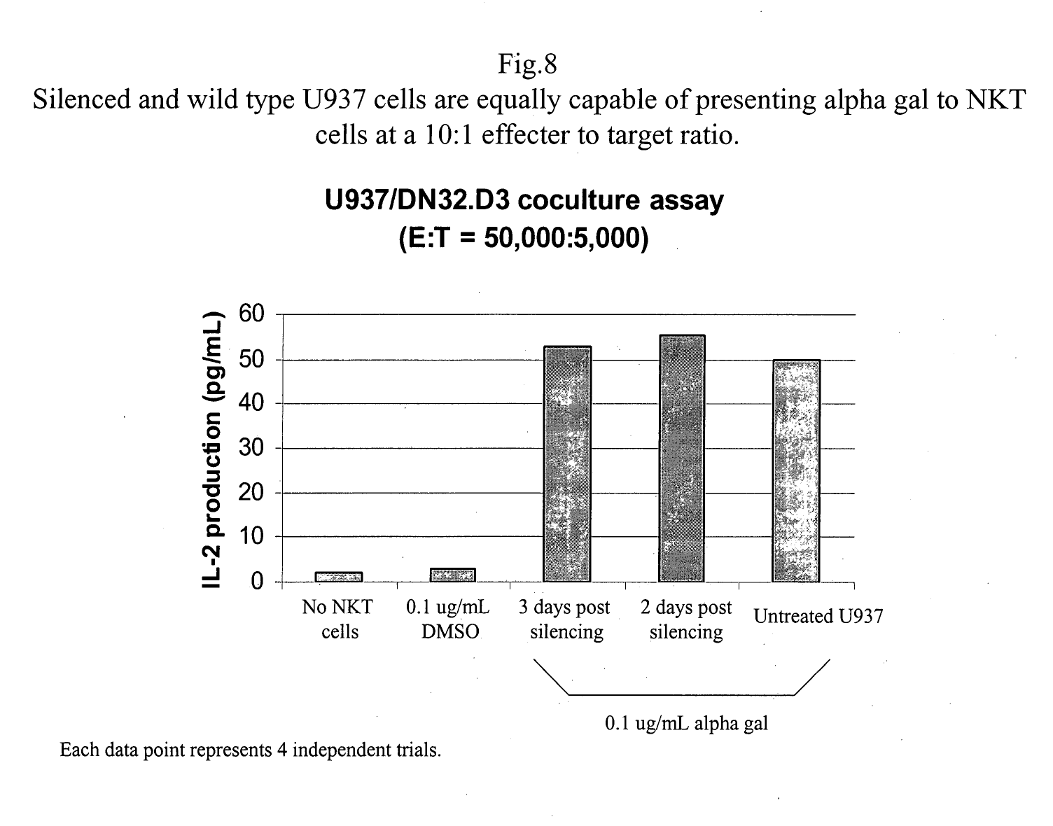

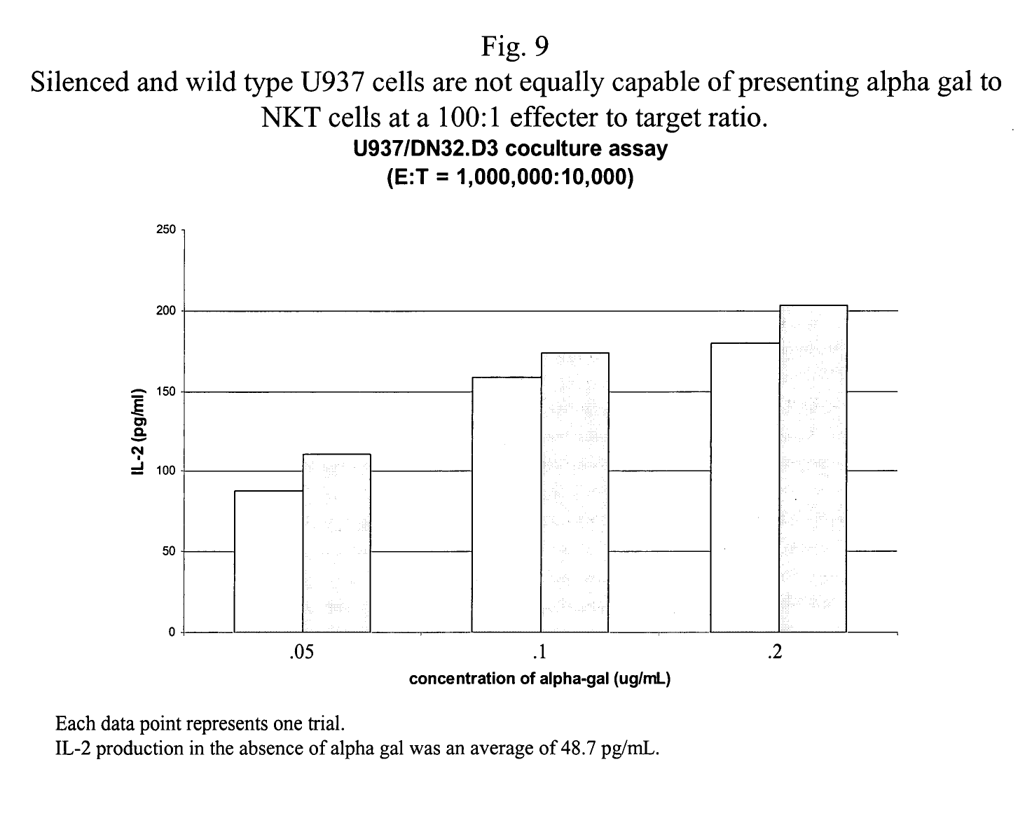

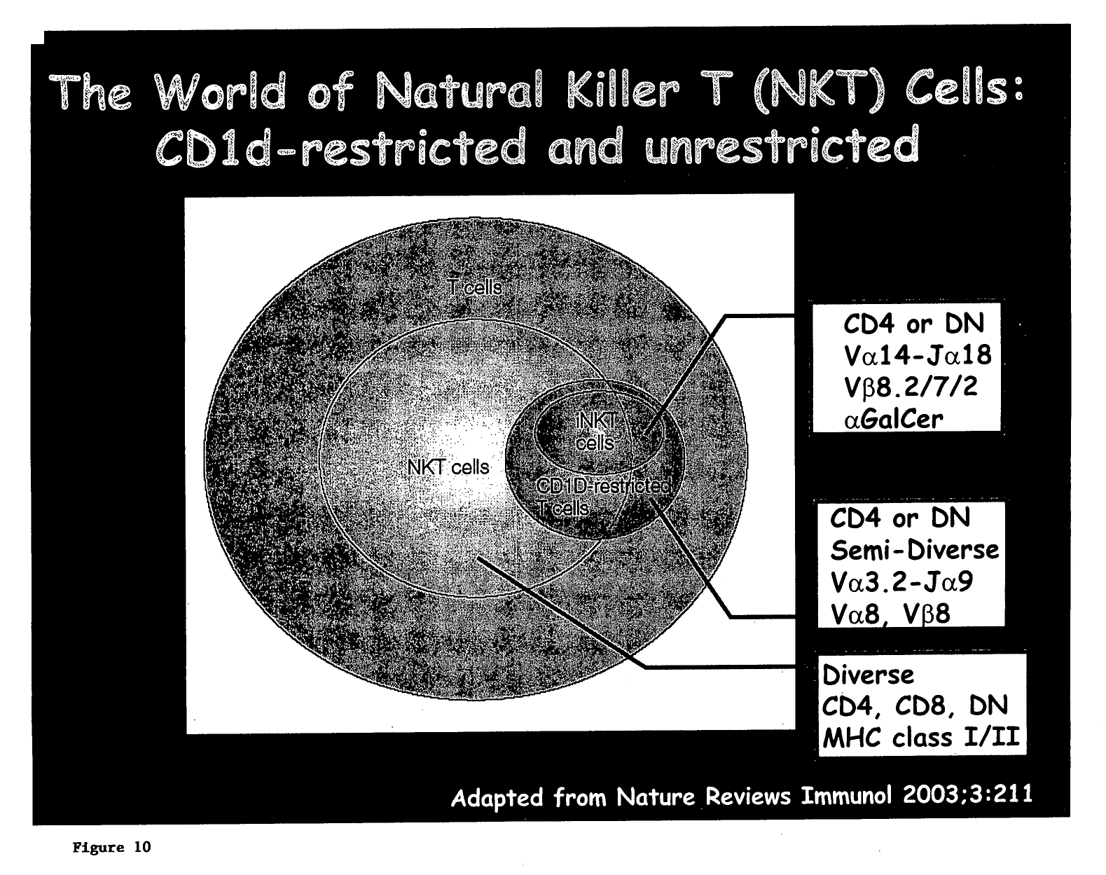

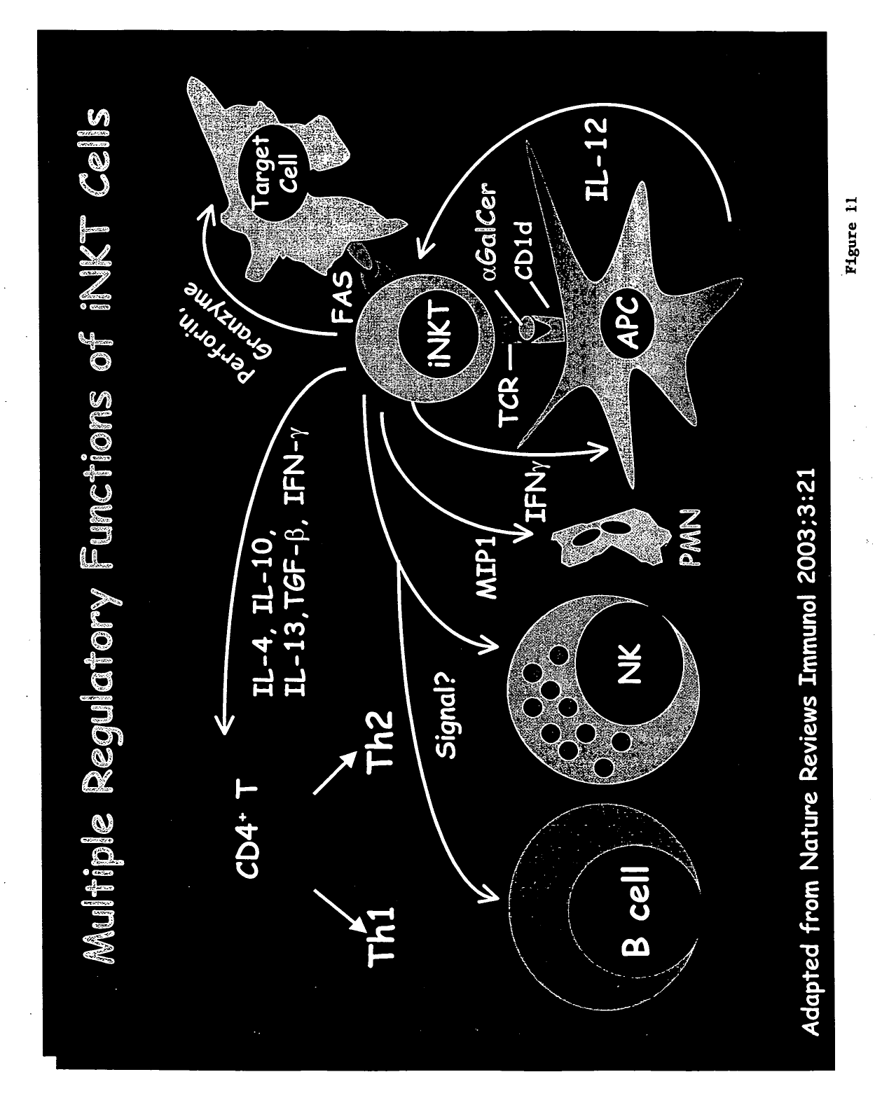

The effect of MTPp gene deletion on the clinicopathologic outcome of oxazolone-induced colitis was examined. This model of colitis has recently been shown to be mediated by CD1d and CD1d-restricted iNKT cells (Heller, F., Fuss, I. J., Nieuwenhuis, E., Blumberg, R. S. & Strober, W. Immunity. 17, 629-638 (2002)). Whereas MTP^{flox/flox}Mx1Cre mice experienced severe colitis in association after administration of the hapten oxazolone, as manifest by profound weight loss (FIG. 4A, panel b, closed squares) and pathological evidence of mucosal ulcerations and infiltration of intestinal tissues by inflammatory cells (FIG. 4B, panel c), MTPp^{Δ/Δ} mice in which the MTPp gene had been deleted by administration of pIpC exhibited minimal weight loss, which was identical to that of the MTP^{flox/flox}Mx1Cre mice exposed to the ethanol control (FIG. 4A, panel b, closed circles) and little histologic evidence of colitis (FIG. 4B, panel d). A semiquantitative estimate of colitis severity confirmed these results (FIG. 4C, panel b). Specifically, MTP^{flox/flox}Mx1Cre mice exhibited little evidence of colitis in response to the administration of oxazolone when pretreated with pIpC. Given that colitis was not ameliorated in WT B6 mice exposed to oxazolone in the presence of pIpC administration at the same schedule administered to the MTP^{flox/flox}Mx1Cre mice (FIG. 4A-C), these results indicate that the protection observed in the MTPp^{Δ/Δ} mice was not simply due to the effects of the pIpC treatment.

Other embodiments are within the following claims.

1. A method of inhibiting CD1-mediated inflammation, comprising contacting a CD1-expressing cell with an inhibitor of microsomal triglyceride transfer protein (MTP), wherein activation of a CD1-restricted T cell is reduced following contact of said CD1-expressing cell with an inhibitor of MTP.

2. The method of claim 1, wherein said CD1-expressing cell is an antigen presenting cell.

3. The method of claim 2, wherein said antigen presenting cell is selected from the group consisting of a B cell, a monocyte, a macrophage, a dendritic cell, a hepatocyte, and an epithelial cell.

4. The method of claim 1, wherein said CD1-expressing cell is an epithelial cell.

5. The method of claim 1, wherein said CD1-expressing cell is an intestinal epithelial cell.

6. The method of claim 1, wherein said CD1-expressing cell is a CD1-d expressing cell.

7. The method of claim 6, wherein said CD1-d expressing cell expresses a natural killer receptor or an invariant T cell receptor.

8. The method of claim 7, wherein said invariant T cell receptor comprises human Vα24Jαa15.

9. A method of inhibiting CD1-mediated antigen presentation, comprising contacting a CD1-expressing cell with an inhibitor of microsomal triglyceride transfer protein (MTP), wherein the amount of lipid associated with CD1 or the amount of binding of MTP to CD1 is reduced in the presence of said inhibitor compared to the amount in the absence of said inhibitor.

10. A method of inhibiting a symptom of a CD1-mediated immunopathology, comprising administering to a mammal a MTP inhibitor, wherein said symptom is reduced following administration.

11. The method of claim 10, wherein said immunopathology is an autoimmune disorder.

12. The method of claim 10, wherein said immunopathology is selected from the group consisting of colitis or hepatitis.

13. A method of inhibiting an association of MTP and CD1, comprising contacting a cell expressing said MTP and said CD1 with an MTP-binding compound, wherein said association is reduced in the presence of said compound compared to that in the absence of said compound.

14. A method of inhibiting an association of MTP and CD1, comprising contacting a cell expressing said MTP and said CD1d with an CD1-binding compound, wherein said association is reduced in the presence of said compound compared to that in the absence of said compound.

15. A method of reducing lipidation of CD1d in a cell, comprising contacting said cell with, a compound which inhibits binding of a alpha 1, alpha 2, or alpha 3 domain of CD1d with MTP.

16. A method inhibiting inflammation, comprising administering to an inflamed tissue a microsomal triglyceride transfer protein (MTP) inhibitor.

17. The method of claim 16, wherein said tissue is intestinal epithelial tissue.

18. The method of claim 16, wherein said inflammation is skeletal inflammation, gastrointestinal inflammation, cardiovascular inflammation, pulmonary inflammation, or neurological inflammation.

19. The method of claim 18, wherein said gastrointestinal inflammation is colitis.

20. The method of claim 16, wherein said tissue is pulmonary tissue, liver tissue, intestinal tissue or dermal tissue.

21. The method of claim 16, wherein said MTP inhibitor is a compound according to Formula I: 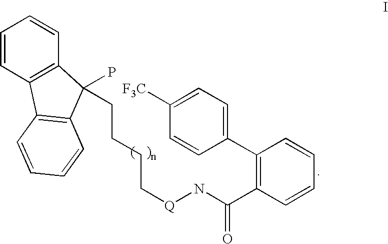
wherein n is zero or 1;
P is 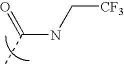
or a 5- or 6-membered heterocycle selected from the group consisting of: 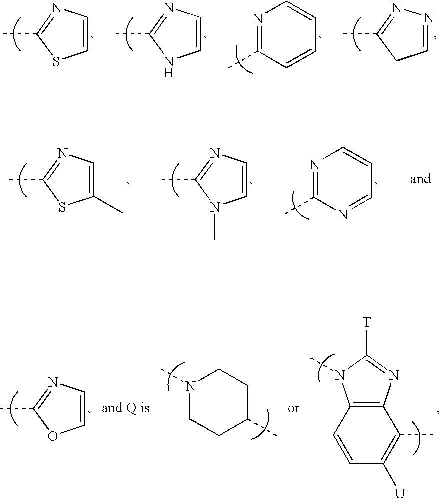
wherein T and U are, independently, hydrogen or lower alkyl.

22. The method of claim 16, wherein said MTP inhibitor is a compound according to Formula I: 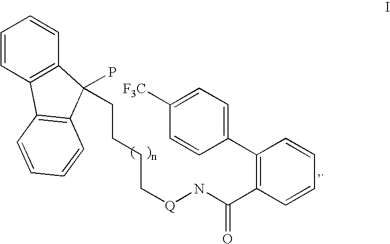
wherein n is zero or 1;
P is 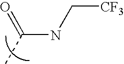
or a 5- or 6-membered heterocycle selected from the group consisting of: 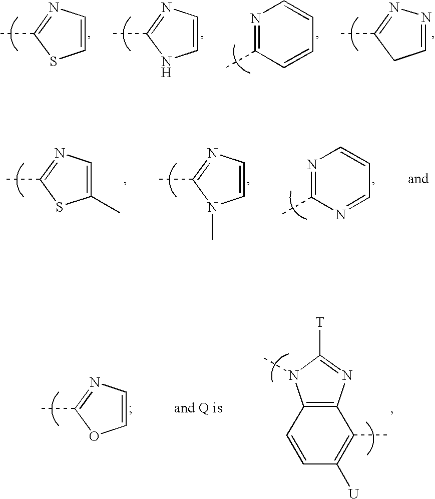
wherein T and U are, independently, hydrogen or lower alkyl.

23. The method of claim 22, wherein P is

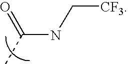

24. The method of claim 16, wherein said MTP inhibitor is a MTP antisense nucleic acid or a MTP siRNA.

25. The method of claim 24, wherein the siRNA comprises the nucleic acid sequence of SEQ ID NO:1 or SEQ ID NO:2.

26. A method of preventing or alleviating a symptom of an inflammatory disorder comprising identifying a subject suffering from of at risk of developing said inflammatory disorder and administering to said subject a microsomal triglyceride transfer protein (MTP) inhibitor.

27. The method of claim 26, wherein said MTP inhibitor is a compound according to Formula I: 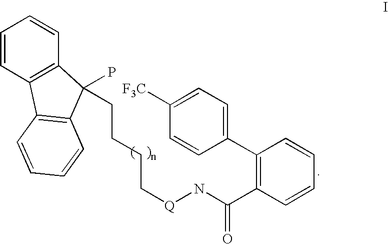
wherein n is zero or 1;
P is 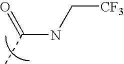
or a 5- or 6-membered heterocycle selected from the group consisting of: 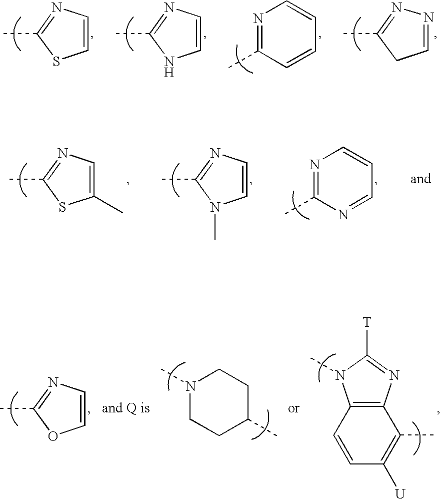
wherein T and U are, independently, hydrogen or lower alkyl.

28. The method of claim 26, wherein said MTP inhibitor is a compound according to Formula I: 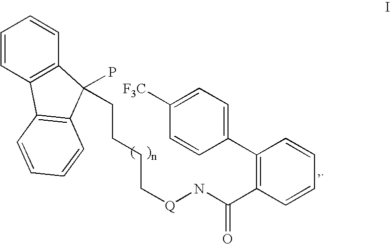
wherein n is zero or 1;
P is 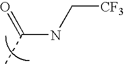
or a 5- or 6-membered heterocycle selected from the group consisting of: 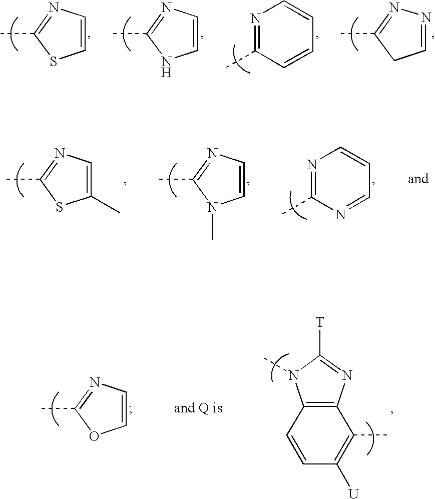
wherein T and U are, independently, hydrogen or lower alkyl.

29. The method of claim 28, wherein P is

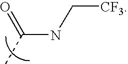

30. The method of claim 26, wherein said MTP inhibitor is a MTP antisense nucleic acid or MTP siRNA.

31. The method of claim 26, wherein the siRNA comprises the nucleic acid sequence of SEQ ID NO:1 or SEQ ID NO:2.

32. The method of claim 26, wherein said inflammatory disorder is a skeletal inflammatory disorder, a gastrointestinal inflammatory disorder, an oral inflammatory disorder, a cardiovascular inflammatory disorder, a pulmonary inflammatory disorder, an auto-immune disorder or a neurological inflammatory disorder.

33. The method of claim 32, wherein said gastrointestinal disorder is inflammatory bowel disease, Crohn's disease or colitis.

34. A method of inhibiting tissue inflammation comprising contacting a cell with a MTP inhibitor in an amount that inhibits the production of an inflammatory cytokine.

35. The method of claim 34, wherein said MTP inhibitor is a compound according to Formula I: 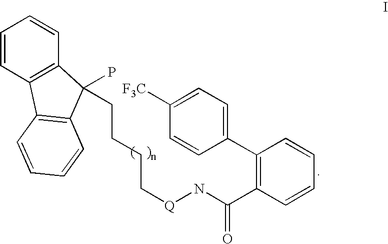
wherein n is zero or 1;
P is 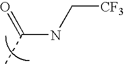
or a 5- or 6-membered heterocycle selected from the group consisting of: 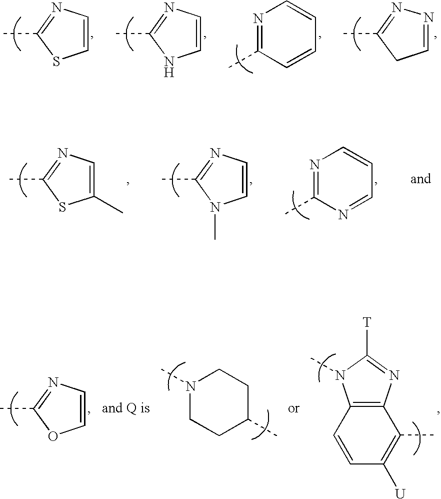
wherein T and U are, independently, hydrogen or lower alkyl.

36. The method of claim 34, wherein said MTP inhibitor is a compound according to Formula I: 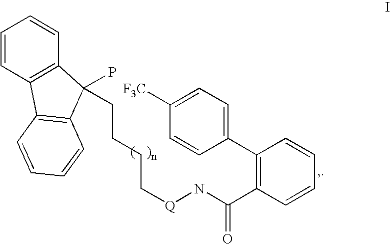
wherein n is zero or 1;
P is 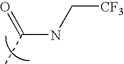
or a 5- or 6-membered heterocycle selected from the group consisting of: 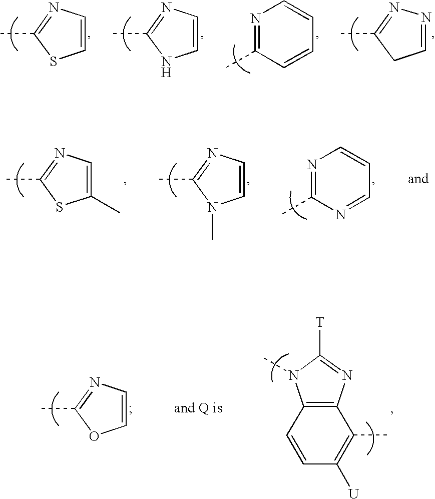
wherein T and U are, independently, hydrogen or lower alkyl.

37. The method of claim 34, wherein P is

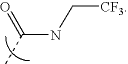

38. The method of claim 34, wherein said MTP inhibitor is a MTP antisense nucleic acid or a MTP siRNA.

39. The method of claim 38, wherein the siRNA comprises the nucleic acid sequence of SEQ ID NO:1 or SEQ ID NO:2.

40. The method of claim 34, wherein said cell is a hepatocyte or an epithelial cell.

41. The method of claim 40, wherein said epithelial cell is an intestinal epithelial cell.

42. The method of claim 34, wherein said inflammatory cytokine is interferon, interleukin or tumor necrosis factor alpha.

43. A method of inhibiting tissue inflammation, comprising contacting a cell with a MTP inhibitor in an amount that inhibits T-cell activation.

44. The method of claim 43, wherein said MTP inhibitor is a compound according to Formula I: 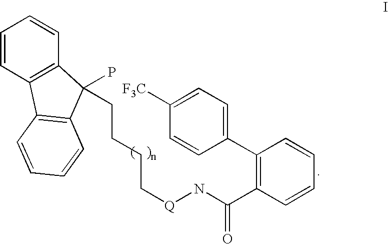
wherein n is zero or 1;
P is 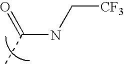
or a 5- or 6-membered heterocycle selected from the group consisting of: 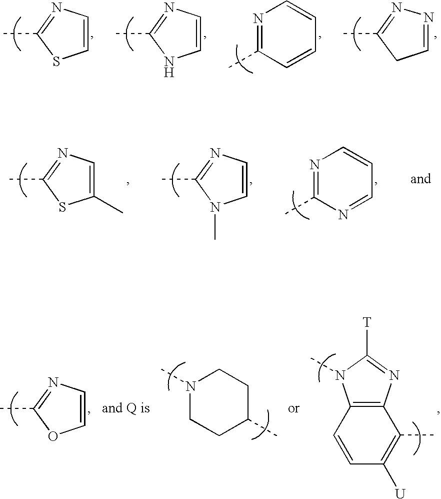
wherein T and U are, independently, hydrogen or lower alkyl.

45. The method of claim 43, wherein said MTP inhibitor is a compound according to Formula I: 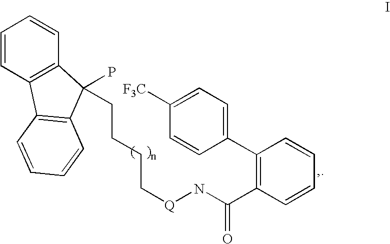
wherein n is zero or 1;
P is 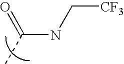
or a 5- or 6-membered heterocycle selected from the group consisting of: 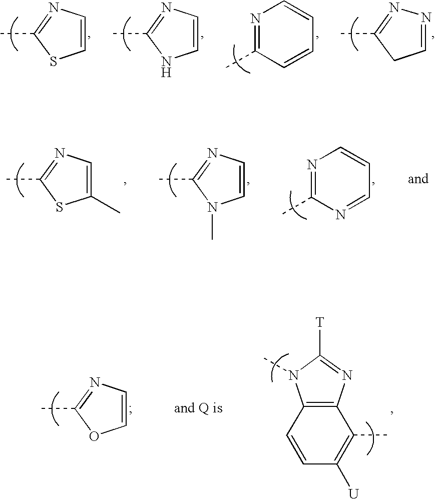
wherein T and U are, independently, hydrogen or lower alkyl.

46. The method of claim 44, wherein P is

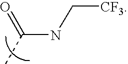

47. The method of claim 43, wherein said MTP inhibitor is a MTP antisense nucleic acid or MTP siRNA.

48. The method of claim 47, wherein the siRNA comprises the nucleic acid sequence of SEQ ID NO:1 or SEQ ID NO:2.

49. The method of claim 43, wherein said cell is a hepatocyte or an epithelial cell.

50. The method of claim 49, wherein said epithelial cell is an intestinal epithelial cell.
